(19) United States
(12) Patent Application Publication
(10) Pub. No.: US 2006/0276505 A1
(43) Pub. Date: Dec. 7, 2006

(54) Acylhydrazine P2X7 antagonists and uses thereof

(76) Inventors: Derek W. Nelson, Highland Park, IL (US); Michael F. Jarvis, Vernon Hills, IL (US); William A. Carroll, Evanston, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/400,492

(22) Filed: Apr. 7, 2006

Related U.S. Application Data
(60) Provisional application No. 60/670,208, filed on Apr. 11, 2005.

(51) Int. Cl. A61K 31/47; C07D 215/12

(52) U.S. Cl. 514/311; 546/176

(57) ABSTRACT

The present invention discloses a compound of formula (I) 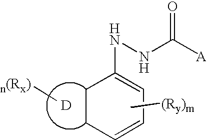
or a pharmaceutically acceptable salt or prodrug thereof, wherein D, A, m, n, Rₓ and R_{y} are defined in the description. The present invention also relates to pharmaceutical compositions of compounds of formula (I), which are useful for treating a disorder selected from the group consisting of chronic inflammatory pain, neuropathic pain, inflammation, neurodegeneration, depression and promoting neuroregeneration.
The present invention also relates to a method for treating pain, neuropathic pain, inflammation, chronic inflammatory pain, neurodegeneration, depression and promoting neuroregeneration in a mammal using compounds of formula (II), 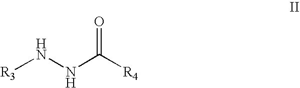
a pharmaceutically acceptable salt, ester, amide or prodrug thereof, wherein R₃ and R₄ are defined in the description.

This application claims priority to the provisional application Ser. No. 60/670,208 filed on Apr. 11, 2005.

### TECHNICAL FIELD

The present invention relates to compounds of formula (I) that are P2X₇ antagonists and are useful for treating pain, neuropathic pain, inflammation, neurodegeneration, depression and for promoting neuroregeneration. The present invention also relates to the use of compounds of formula (II) to treat or prevent pain, neuropathic pain, inflammation, neurodegeneration, depression and to promote neuroregeneration.

### BACKGROUND OF THE INVENTION

P2X receptors are ionotropic receptors activated by ATP. The importance of P2X receptors in nociception is underscored by the variety of pain states in which this endogenous ligand can be released. Of the seven P2X receptors, the P2X₇ is distinguished by its ability to form a large pore upon prolonged or repeated agonist stimulation. It is partially activated by saturating concentrations of ATP, whereas it is fully activated by the synthetic ATP analog benzoylbenzoic ATP (BzATP) (Bianchi et al., Eur. J. Pharmacol. Vol. 376, pages 127-138, 1999). The P2X₇ receptor is expressed by presynaptic terminals in the central and peripheral nervous systems, antigen-presenting cells including macrophages, human epidermal Langerhans' cells, microglial cells and a number of tumor cell lines of varying origin (Jacobson K A, et al. “Adenosine and Adenine Nucleotides: From Molecular Biology to Integrative Physiology”. L. Belardinelli and A. Pelleg (eds.), Kluwer, Boston, pages 149-166, 1995).

Recent studies demonstrated the participation of P2X₇ receptors in the modulation of electrical stimulation and ATP-evoked GABA and glutamate release from mouse hippocampal slices (Papp et al., Neuropharmacology and Neurotoxicology Vol. 15, pages 2387-2391, 2004)). In the central nervous system, the P2X₇ receptor is predominately expressed by microglia, the resident macrophages of the brain. On glial cells, the P2X₇ receptor has been shown to mediate release of glutamate (Anderson C. et al. Drug Dev. Res. Vol. 50. page 92, 2000). Upregulation of the P2X₇ receptor, most likely on activated microglia, was reported in association with ischemic damage and necrosis induced by occlusion of middle cerebral artery in rat brain (Collo G. et al. Neuropharmacology, Vol. 36, pages 1277-1283, 1997). Recent studies indicate a role of the P2X₇ receptor in the generation of superoxide in microglia, and upregulation of P2X₇ receptors around β-amyloid plaques in a transgenic mouse model for Alzheimer's disease (Parvathenani et al., J. Biol. Chemistry, Vol. 278, pages 13300-13317, 2003) and in multiple sclerosis lesions from autopsy brain sections (Narcisse et al., Glia, Vol. 49, pages 245-258 (2005).

Activation of the P2X₇ receptor on cells of the immune system (macrophages, mast cells and lymphocytes) leads to release of interleukin-1β (IL-1β), giant cell formation, degranulation, and L-selectin shedding. ATP has been shown to increase local release and process of IL-1β following lipopolysaccharide S (LPS) intraperitoneal injections in rats through a X₇ receptor mediated mechanism (Griffiths et al., J. Immunology Vol. 154. pages 2821-2828 (1995); Solle et al., J. Biol. Chemistry Vol. 276, pages 125-132, (2001)).

Oxidized ATP (oATP), a nonselective and irreversible P2X₇ antagonist, was recently reported to possess peripherally mediated antinociceptive properties in inflamed rats (Dell'Antonio et al. Neuroscience Lett., Vol. 327, pages 87-90, 2002). Activation of P2X₇ receptors localized on presynaptic terminals in the central and peripheral nervous systems (Deuchars et al J. Neuroscience, Vol. 21, pages 7143-7152, 2001) induced release of the excitatory amino acid neurotransmitter glutamate.

Studies from mice lacking P2X₇ receptor rsulted in absence of inflammatory and neuropathic hypersensitivity to mechanical and thermal stimuli, indicating a link between a P2X₇ purinoceptor gene and inflammatory and neuropathic pain (Chessell et al., Pain, Vol 114, pages 386-396 (2005)).

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, 2004).

Taken together, these findings indicate that compounds acting at the P2X₇ receptor may have utility in the treatment of pain, inflammatory processes, and degenerative conditions associated with disease states such as rheumatoid arthritis, osteoarthritis, psoriasis, allergic dermatitis, asthma, chronic obstructive pulmonary disease, airways hyper-responsiveness, septic shock, glomerulonephritis, irritable bowel disease, Crohn's disease, ulcerative colitis, atherosclerosis, growth and metastases of malignant cells, myoblastic leukaemia, diabetes, Alzheimer's disease, multiple sclerosis, meningitis, osteoporosis, burn injury, ischemic heart disease, stroke and varicose veins.

In view of the above facts, there is a need for selective P2X₇ antagonist that can be efficiently used in preventing, treating, or ameliorating states as neuropathic pain, chronic inflammatory pain, inflammation and neurodegenerative conditions associated with several progressive CNS disorders, including, but not limited to, Alzheimer's disease, Parkinson's disease, depression, amyotrophic lateral sclerosis, Huntington's disease, dementia with Lewy bodies, multiple sclerosis as well as diminished CNS function resulting from traumatic brain injury.

### SUMMARY OF THE INVENTION

In its principal embodiment, the present invention relates to a compound of formula (I) 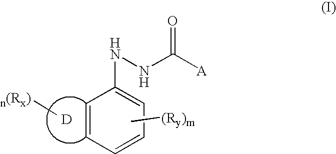
or a pharmaceutically acceptable salt or prodrug thereof, wherein
D is a five or six-membered heteroaryl ring selected from the group consisting of pyridine, pyridizine, pyrimidine, pyrazine, pyrazole, isothiazole, thiazole, isoxazole, oxazole and furazan;
m is 0, 1, 2 or 3;
n is 0, 1, 2, 3 or 4;
Rₓ and R_{y} are independently selected from the group consisting of alkyl, alkenyl, halogen, nitro, cyano, haloalkyl, —C(O)alkyl, —C(O)OH, —C(O)Oalkyl, —C(O)NH₂, —C(O)N(H)(alkyl), —C(O)N(alkyl)₂ and -G₁-G₂-G₃;
G₁ at each occurrence is independently selected from the group consisting of a bond O, S and —N(R₁₀₁)—;
G₂ at each occurrence is independently selected from the group consisting of a bond, alkyl and -alkyl-N(R₁₀₁)-alkyl-;
G₃ at each occurrence is independently selected from the group consisting of hydrogen, alkyl, —N(R₁₀₂)(R₁₀₃), and —O(R₁₀₂);
R₁₀₁ at each occurrence is independently selected from the group consisting of hydrogen, alkyl, alkenyl, haloalkyl, haloalkenyl, hydroxyalkyl, and alkoxyalkyl;
R₁₀₂ at each occurrence is independently selected from the group consisting of hydrogen alkyl and haloalkyl;
R₁₀₃ at each occurrence is selected from the group consisting of hydrogen, alkyl, alkenyl, haloalkyl, hydroxyalkyl, alkxoyalkyl, -alkyl-NH₂, -alkyl-N(H)(alkyl), -alkyl-N(alkyl)₂, —C(O)alkyl, and -alkyl-C(O)O(alkyl);
alternatively, R₁₀₂ and R₁₀₃, together with the nitrogen atom to which they are attached, form a saturated four to nine membered heterocyclic ring; wherein the heterocyclic ring may comprise a second ring heteroatom selected from the group consisting of nitrogen and oxygen, and the ring is substituted with 0, 1, 2 or 3 substituents selected from the group consisting of —OH, halogen, alkyl, alkenyl, hydroxyalkyl, -alkyl-NH₂, -alkyl-N(H)(alkyl), -alkyl-N(alkyl)₂, and —N(H)(—CH₂CH₂OH);
A is R₁ or -L₁-R₂;
L₁ is C₁-C₆ alkylenyl substituted with 0, 1 or 2 substituents selected from the group consisting of alkoxy, halogen, haloalkyl, and R_{c};
R₁ is selected from the group consisting of cycloalkenyl, cycloalkyl and heterocycle; wherein each RI is independently substituted with 0, 1, 2, 3, 4 or 5 substituents independently selected from the group consisting of alkenyl, alkyl, alkynyl, halogen, haloalkyl, nitro, oxo, R_{c}, -alkylR_{c}, -alkylOR_{c} and -G₁-G₂-G₃;
R₂ is selected from the group consisting of heteroaryl, aryl, cycloalkenyl and cycloalkyl;
wherein each R₂ is independently substituted with 0, 1 or 2 substituents independently selected from the group consisting of alkyl, haloalkyl, -G₁-G₂-G₃ and R_{c}; and
R_{c} at each occurrence is independently selected from the group consisting of cycloalkyl, cycloalkenyl, heterocycle, aryl and hetroaryl; wherein each R_{c} is independently substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)OH, —C(O)Oalkyl, —C(O)NH₂, —C(O)N(H)(alkyl) and —C(O)N(alkyl)₂.
The invention also relates to a method for inhibiting P2X₇ activity comprising administering to a patient in need of such treatment a therapeutically effective amount of a compound of formula I, or a therapeutically acceptable salt, solvate, prodrug, salt of a prodrug, or combination thereof.

The invention also relates to a pharmaceutical composition comprising a therapeutically effective amount of a compound of formula I, or a therapeutically acceptable salt, solvate, prodrug, salt of a prodrug, or combination thereof, and a pharmaceutically acceptable carrier, useful for treating a disorder selected from the group consisting of chronic inflammatory pain, neuropathic pain, inflammation, neurodegeneration, depression and promoting neuroregeneration, comprising administering to a patient in need of such treatment.

The present invention also contemplates a method of treating neuropathic pain, chronic inflammatory pain, inflammation, neurodegeneration, depression and of promoting neuroregeneration comprising administering a therapeutically effective amount of a selective P2X₇ receptor antagonist of formula (II), 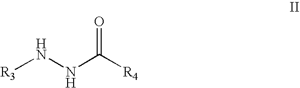
a pharmaceutically acceptable salt, ester, amide or prodrug thereof, wherein
R₃ is selected from the group consisting of alkyl, cycloalkyl, cycloalkenyl, heterocyclealkyl, aryl, and heteroaryl; wherein the cycloalkyl, cycloalkenyl, heterocyclealkyl, aryl and heteroaryl are independently substituted with 0, 1, 2, 3, 4 or 5 substituents independently selected from the group consisting of alkyl, alkenyl, halogen, nitro, cyano, haloalkyl, -G₁-G₂-G₃, —C(O)alkyl, —C(O)OH and —C(O)Oalkyl;
R₄ is selected from the group consisting of alkyl, alkenyl, alkynyl, cycloalkenyl, cycloalkyl and heterocycle; wherein the alkyl is substituted with 0, 1 or 2 substituents independently selected from the group consisting of Rₐ and R_{b}, and wherein each of the cycloalkenyl, cycloalkyl and heterocycle is independently substituted with 0, 1, 2, 3, 4, or 5 substituents independently selected from the group consisting of alkenyl, alkyl, alkynyl, halogen, haloalkyl, nitro, oxo, aryloxy, -G₁-G₂-G₃, —S(O)₂alkyl, —C(O)alkyl, R_{b}, -alkylR_{b}, and -alkylOR_{b}; wherein the aryl moiety of the aryloxy is substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)OH, —C(O)Oalkyl, —C(O)NH₂, —C(O)N(H)(alkyl) and —C(O)N(alkyl)₂;
Rₐ at each occurrence is independently selected from the group consisting of —OH, alkoxy, —OR_{b}, —O-alkyl-R_{b}, —S(alkyl), —SR_{b}, —S(O)₂alkyl, —S(O)₂R_{b}, —C(O)alkyl, —C(O)R_{b}, —N(H)C(O)alkyl, —N(H)C(O)R_{b}, —N(H)S(O)₂alkyl, —N(H)S(O)₂R_{b}, —C(O)N(H)alkyl and —C(O)N(H)R_{b};
R_{b} at each occurrence is independently selected from the group consisting of cycloalkyl, cycloalkenyl, heterocycle, aryl and hetroaryl; wherein each R_{b} at each occurrence is independently substituted with 0, 1, 2, 3, 4 or 5 substituents independently selected from the group consisting of alkyl, alkenyl, halogen, nitro, cyano, haloalkyl, —OH, alkoxy, aryloxy, haloalkoxy, —S(O)₂alkyl, —NH₂, —N(H)(alkyl), —N(alkyl)₂, —N(H)S(O)₂alkyl, —N(alkyl)S(O)₂alkyl, —N(H)C(O)alkyl, —N(alkyl)C(O)alkyl, —C(O)alkyl, —C(O)NH₂, —C(O)N(H)(alkyl), —C(O)N(alkyl)₂, —C(O)OH and —C(O)Oalkyl; wherein the aryl moiety of the aryloxy is substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)NH₂, —C(O)N(H)(alkyl), —C(O)N(alkyl)₂, —C(O)OH and —C(O)Oalkyl;
G₁ at each occurrence is independently selected from the group consisting of a bond O, S and —N(R₁₀₁)—;
G₂ at each occurrence is independently selected from the group consisting of a bond, alkyl and -alkyl-N(R₁₀₁)-alkyl-;
G₃ at each occurrence is independently selected from the group consisting of hydrogen, alkyl, —N(R₁₀₂)(R₁₀₃), and —O(R₁₀₂);
R₁₀₁ at each occurrence is independently selected from the group consisting of hydrogen, alkyl, alkenyl, haloalkyl, haloalkenyl, hydroxyalkyl, and alkoxyalkyl;
R₁₀₂ at each occurrence is independently selected from the group consisting of hydrogen alkyl and haloalkyl; and
R₁₀₃ at each occurrence is independently selected from the group consisting of hydrogen, alkyl, alkenyl, haloalkyl, hydroxyalkyl, alkxoyalkyl, -alkyl-NH₂, -alkyl-N(H)(alkyl), -alkyl-N(alkyl)₂, —C(O)alkyl, and -alkyl-C(O)O(alkyl);
alternatively, R₁₀₂ and R₁₀₃, together with the nitrogen atom to which they are attached, form a saturated four to nine membered heterocyclic ring; wherein the heterocyclic ring may comprise a second ring heteroatom selected from the group consisting of nitrogen and oxygen, and the ring is substituted with 0, 1, 2 or 3 substituents selected from the group consisting of —OH, halogen, alkyl, alkenyl, hydroxyalkyl, -alkyl-NH₂, -alkyl-N(H)(alkyl), -alkyl-N(alkyl)₂, and —N(H)(—CH₂CH₂OH).

### DETAILED DESCRIPTION OF THE INVENTION

All references contained herein are fully incorporated by reference.

### a) DEFINITION OF TERMS

The term “alkenyl” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkoxyalkyl” as used herein, refers to an alkyl group, as defined herein, in which one, two or three hydrogen atoms are replaced by alkoxy, as defined herein.

The term “alkyl” as used herein, means a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, 2-methylbutyl, 3-methylbutyl, 1,1-dimethypropyl, 2,2,-dimethylpropyl, 3,3-dimethylpropyl, 1-ethylpropyl, 3-ethylpropyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “C₁-C₆ alkylenyl” as used herein, means a divalent group derived from a straight or branched chain hydrocarbon of from 1 to 6 carbon atoms. Representative examples of C₁-C₆ alkylenyl include, but are not limited to, —CH₂—, —CH(CH₃)—, —CH(CH(CH₃)₂)—, —C(CH₃)₂—, —CH₂CH₂—, —CH₂CH₂CH₂—, —CH₂CH₂CH₂CH₂—, —CH₂CH₂CH₂CH₂CH₂— and —CH₂CH(CH₃)CH₂—.

The term “alkynyl” as used herein, refers to a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, 1-butynyl and 3-butynyl.

The term “aryl” as used herein, means a phenyl group, a naphthyl group or an anthracenyl group. The aryl groups of the present invention are appended to the parent moiety through any substitutable atoms in the group and can be unsubstituted or substituted.

The term “aryloxy” as used herein, means an aryl group, as defined herein, appended to the parent molecular moiety through an oxygen atom. Representative examples of aryloxy include, but not limited to, phenoxy.

The term “cyano” as used herein, refers to —CN.

The term “cycloalkyl” or “cycloalkane” as used herein, refers to a saturated monocyclic hydrocarbon ring system having three to eight carbon atoms and zero heteroatom. Examples of monocyclic ring systems include cyclopropyl, cyclobutyl, cyclopentyl, cyclohexyl, cycloheptyl, and cyclooctyl. The monocyclic cycloalkyl of the present invention may contain one or two bridges. The term “bridge” refers to a connection between two of the non-adjacent carbon atoms connected by an alkylene bridge between one and three additional carbon atoms. Representative examples of monocyclic cycloalky that contain such bridge or bridges include, but are not limited to, bicyclo[2.2.1]heptan-1-yl, bicyclo[2.2.1]heptan-2-yl, bicyclo[2.2.1]heptan-1-yl, bicyclo[3.1.1]heptan-6-yl, bicyclo[2.2.2]octan-1-yl and adamantyl. The term “cycloalkyl” of the present invention also include a spiroalkyl, a bicyclic cycloalkyl or tricyclic cycloalkyl. The term “spiroalkyl” as used herein refers to a monocyclic ring substituted with a straight chained alkylene group wherein two carbon atoms of the alkylene group are attached to one carbon atom of the monocyclic cycloalkyl group. Representative example of the spiroalky includes, but is not limited to, spiro[2,5]octyl. The bicyclic cycloalkyl of the present invention refers to a monocyclic cycloalkyl ring fused to another monocyclic cycloalkyl group, as defined herein, or an aryl group as defined herein. Representative examples of the bicyclic cycloalkyl include, but are not limited to, indan-2-yl, 4a(2H)octahydronaphthalenyl, 4a(2H)decahydronaphthalenyl, 1,2,3,4-tetrahydronaphthalen-1-yl. The bicyclic cycloalkyl groups of the present invention may have two of the non-adjacent carbon atoms connected by an alkylene bridge between one and three additional carbon atoms. Representative examples of the bicyclic cycloalkyl groups that contain such connection between two non-adjacent carbon atoms include, but not limited to, octahydro-2,5-methanopentalenyl and 3a(1H)-octahydro-2,5-methanopentalenyl. The tricyclic cycloalkyl group of the present invention refers to a bicyclic cycloalkyl ring, as defined hereinabove, fused to another monocyclic cycloalkyl group, as defined herein, or an aryl group as defined herein. Representative example of the tricyclic cycloalkyl group includes, but is not limited to, dodecahydro-1H-fluoren-9-yl. The monocyclic, sprioalkyl, bicyclic and tricyclic cycloalkyl groups of the present invention can be unsubstituted or substituted, and are connected to the parent molecula moiety through any substitutable carbon atom of the cycloalkyl moiety or cycloalkyl moieties in the group.

The term “cycloalkenyl” or “cycloalkene” as used herein, refers to a non-aromatic, partially unsaturated, monocyclic hydrocarbon ring system, having 4, 5, 6, 7 or 8 carbon atoms and zero heteroatom. The 4-membered ring systems have one double bond, the 5-or 6-membered ring systems have one or two double bonds, and the 7- or 8-membered ring systems have one, two or three double bonds. Representative examples of cycloalkenyl groups include, but not limited to, cyclobutenyl, cyclopentenyl, cyclohexenyl, cycloheptenyl and cyclooctenyl. The monocyclic cycloalkenyl of the present invention may contain one or two bridges. The term “bridge” refers to a connection between two of the non-adjacent carbon atoms connected by an alkylene bridge between one and three additional carbon atoms. Representative examples of monocyclic cycloalkenyls that contain such bridge or bridges include, but are not limited to, bicyclo[2.2.1]hepten-5-yl. The cycloalkenyl groups of the present invention can be unsubstituted or substituted, and are attached to the parent molecular moiety through any substitutable carbon atom of the group.

The term “halo” or “halogen” as used herein, means —Cl, —Br, —I or —F.

The term “haloalkenyl” as used herein, refers to an alkenyl group, as defined herein, in which one, two or three hydrogen atoms are replaced by halogen.

The term “haloalkoxy” as used herein, refers to an alkoxy group, as defined herein, in which one, two, three, four, five or six hydrogen atoms are replaced by halogen. Representative examples of haloalkoxy include, but are not limited to, chloromethoxy, 2-fluoroethoxy, trifluoromethoxy, 2-chloro-3-fluoropentyloxy, and pentafluoroethoxy.

The term “haloalkoxyalkyl” as used herein, refers to an alkyl group, as defined herein, in which one, two or three hydrogen atoms are replaced by haloalkoxy, as defined herein.

The term “haloalkyl” as used herein, refers to an alkyl group, as defined herein, in which one, two, three, four, five or six hydrogen atoms are replaced by halogen. Representative examples of haloalkyl include, but are not limited to, chloromethyl, 2-fluoroethyl, trifluoromethyl, pentafluoroethyl, 2-chloro-3-fluoropentyl and hexafluoropropyl.

The term “heterocycle” or “heterocyclic” as used herein, refers to a monocyclic or bicyclic, non-aromatic, saturated or partially unsaturated ring system. Monocyclic ring systems are exemplified by a 4-membered ring containing one heteroatom independently selected from oxygen, nitrogen and sulfur; or a 5-, 6-, 7-, or 8-membered ring containing one, two or three heteroatoms wherein the heteroatoms are independently selected from nitrogen, oxygen and sulfur. The 5-membered ring has 0 or 1 double bond. The 6-membered ring has 0, 1 or 2 double bonds. The 7- or 8-membered ring has 0, 1, 2 or 3 double bonds. Representative examples of monocyclic ring systems include, but are not limited to, azetidinyl, azepanyl, azepinyl, diazepinyl, dioxolanyl, dioxanyl, dithianyl, imidazolinyl, imidazolidinyl, isothiazolinyl, isothiazolidinyl, isoxazolinyl, isoxazolidinyl, morpholinyl, 3-oxo-morpholinyl, oxadiazolinyl, oxadiazolidinyl, oxazolinyl, 2-oxo-oxazolinyl, oxazolidinyl, piperazinyl, piperidyl, pyranyl, pyrazolinyl, pyrazolidinyl, pyrrolinyl, pyrrolidinyl, tetrahydrofuryl, tetrahydropyranyl, tetrahydropyridyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, 1,4-diazepanyl and trithianyl. Bicyclic heterocyclic ring systems are exemplified by any of the above monocyclic ring systems fused to a phenyl group, a monocyclic cycloalkenyl group, as defined herein, a monocyclic cycloalkyl group, as defined herein, or an additional monocyclic heterocycle group, as defined herein. Representative examples of bicyclic ring systems include but are not limited to, benzodioxinyl, benzodioxolyl, benzopyranyl, benzothiopyranyl, 2,3-dihydroindol-3-yl, 2,3-dihydrobenzofuran-3-yl, 2,3-dihydrobenzothien-3-yl, 2,3-dihydroisoindol-3-yl, 1,3-dihydro-isobenzofuran-3-yl, 1,3-dihydro-benzo[c]thien-3-yl, tetrahydroisoquinolinyl, tetrahydroquinolinyl, 3-azabicyclo[3.2.0]heptyl, 3,6-diazabicyclo[3.2.0]heptyl, octahydrocyclopenta[c]pyrrolyl, hexahydro-1H-furo[3,4-c]pyrrolyl, and octahydropyrrolo[3,4-c]pyrrolyl. The monocyclic or bicyclic ring systems as defined herein may have two of the non-adjacent carbon atoms connected by a heteroatom selected from nitrogen, oxygen or sulfur, or an alkylene bridge of between one and three additional carbon atoms. Representative examples of monocyclic or bicyclic ring systems that contain such connection between two non-adjacent carbon atoms include, but not limited to, 2-azabicyclo[2.2.2]octyl, 2-oxa-5-azabicyclo[2.2.2]octyl, 2,5-diazabicyclo[2.2.2]octyl, 2-azabicyclo[2.2.1]heptyl, 2-oxa-5-azabicyclo[2.2.1]heptyl, 2,5-diazabicyclo[2.2.1]heptyl, 2-azabicyclo[2.1.1]hexyl, 5-azabicyclo[2.1.1]hexyl, 3-azabicyclo[3.1.1]heptyl, 6-oxa-3-azabicyclo[3.1.1]heptyl, 8-azabicyclo[3.2.1]octyl, 8-azabicyclo[3.2.1]oct-8-yl, 3-oxa-8-azabicyclo[3.2.1]octyl, 1,4-diazabicyclo[3.2.2]nonyl, 1,4-diazatricyclo[4.3.1.1^{3,8}]undecyl, 3,10-diazabicyclo[4.3.1]decyl, or 8-oxa-3-azabicyclo[3.2.1]octyl, octahydro-1H-4,7-methanoisoindolyl, and octahydro-1H-4,7-epoxyisoindolyl. The heterocycle groups of the invention are substituted or unsubstituted, and are connected to the parent molecular moiety through any substitutable carbon or nitrogen atom of the heterocycle moiety or heterocycle moieties in the groups. The nitrogen heteroatom may or may not be quaternized, and the nitrogen or sulfur heteroatom may or may not be oxidized. In addition, the nitrogen containing heterocyclic rings may or may not be N-protected.

The term “heteroaryl” as used herein, refers to an aromatic five- or six-membered ring where at least one atom is selected from the group consisting of N, O, and S, and the remaining atoms are carbon. The five membered rings have two double bonds, and the six membered rings have three double bonds. The term “heteroaryl” also includes bicyclic systems where a monocyclic heteroaryl ring is fused to a phenyl group, a monocyclic cycloalkyl group, as defined herein, a monocyclic cycloalkenyl group, as defined herein, a monocyclic heterocycle group, as defined herein, or an additional monocyclic heteroaryl group. Representative examples of heteroaryl groups include, but not limited to, benzothienyl, benzoxazolyl, benzimidazolyl, benzoxadiazolyl, 6,7-dihydro-1,3-benzothiazolyl, furyl, imidazolyl, imidazo[1,2-a]pyridinyl, indazolyl, indolyl, isoindolyl, isoxazolyl, isoquinolinyl, isothiazolyl, naphthyridinyl, oxadiazolyl, oxazolyl, pyridoimidazolyl, pyridyl, pyridazinyl, pyrimidinyl, pyrazinyl, pyrazolyl, pyrrolyl, quinolinyl, thiazolyl, thienyl, triazolyl, thiadiazolyl, tetrazolyl, 1,2,3,4-tetrahydro-1,8-naphthyridin-2-yl, and 5,6,7,8-tetrahydroquinolin-5-yl. The heteroaryl groups of the present invention can be substituted or unsubstituted, and are connected to the parent molecular moiety through any substitutable carbon or nitrogen atom in the groups. In addition, the nitrogen heteroatom may or may not be quaternized, the nitrogen and the sulfur atoms in the group may or may not be oxidized. Also, the nitrogen containing rings may or may not be N-protected.

The term “heteroatom” as used herein, refers to nitrogen, oxygen or sulfur atom.

The term “hydroxy” or “hydroxyl” as used herein, means an —OH group.

The term “hydroxyalkyl” as used herein, refers to an alkyl group, as defined herein, in which one, two or three hydrogen atoms are replaced by hydroxy, as defined herein.

The term “nitro” as used herein, refers to an —NO₂ group.

The term “oxo” as used herein, refers to a ═O group.

### b) COMPOUNDS AND COMPOSITIONS OF THE INVENTION

Compounds of the invention can have the formula (I) as described above. Compounds of the invention include those in which D is selected from the group consisting of pyridine, pyridizine, pyrimidine, pyrazine, pyrazole, isothiazole, thiazole, isoxazole, oxazole and furazan. More particularly, compounds of formula (I) can include, but are not limited to, compounds wherein D is pyridine. Specific examples are, for example, compounds where D is pyridine and together with the phenyl to which it is attached to can be independently selected from the group consisting of quinoline and isoquinoline derivatives.

Preferred compounds of the present invention are those wherein D and the phenyl group to which it is attached to form an isoquinoline group. These compounds include, but are not limited to those in which A is -L₁-R₂; wherein L₁ is selected form the group consisting of substituted or unsubstituted C1-C6 alkenyl as defined above, and R₂ is selected from the group consisting of heteroaryl, aryl, cycloalkyl and cycloalkenyl, as defined above. The present invention also contemplates compounds in which A is R₁. Preferred compounds contemplated in the present invention include, but are not limited to those in which R₁ is elected form the group consisting g of monocyclic, bicyclic and tricyclic cycloalkyl. Other preferred compounds include monocyclic cycloalkyl, which contain one or two bridges as described in the definitions.

Other preferred compounds of the present invention are those wherein D and the phenyl group to which it is attached to form a quinoline group. These compounds include, but are not limited to those in which A is -L₁-R₂; wherein L₁ is selected form the group consisting of substituted or unsubstituted C1-C6 alkenyl as defined above, and R₂ is selected from the group consisting of heteroaryl, aryl, cycloalkyl and cycloalkenyl, as defined above. The present invention also contemplates compounds in which A is R₁. Preferred compounds contemplated in the present invention include, but are not limited to those in which R₁ is elected form the group consisting of monocyclic, bicyclic and tricyclic cycloalkyl. Other preferred compounds include monocyclic cycloalkyl, which contain one or two bridges as described in the definitions.

Compounds of the invention may exist as stereoisomers wherein, asymmetric or chiral centers are present. These stereoisomers are “R” or “S” depending on the configuration of substituents around the chiral element. The terms “R” and “S” used herein are configurations as defined in IUPAC 1974 Recommendations for Section E, Fundamental Stereochemistry, Pure Appl. Chem., 1976, 45: 13-30. The invention contemplates various stereoisomers and mixtures thereof and are specifically included within the scope of this invention. Stereoisomers include enantiomers and diastereomers, and mixtures of enantiomers or diastereomers. Individual stereoisomers of compounds of the invention may be prepared synthetically from commercially available starting materials which contain asymmetric or chiral centers or by preparation of racemic mixtures followed by resolution well-known to those of ordinary skill in the art.

The invention also provides pharmaceutical compositions comprising a therapeutically effective amount of a compound of formula (I) in combination with a pharmaceutically acceptable carrier. The compositions comprise compounds of the invention formulated together with one or more non-toxic pharmaceutically acceptable carriers. The pharmaceutical compositions can be formulated for oral administration in solid or liquid form, for parenteral injection, for rectal or vaginal administration, and for topical, dermal or transdermal administration.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The term “parenteral,” as used herein, refers to modes of administration, including intravenous, intramuscular, intraperitoneal, intrasternal, subcutaneous, intraarticular injection and infusion.

Pharmaceutical compositions for parenteral injection comprise pharmaceutically acceptable sterile aqueous or nonaqueous solutions, dispersions, suspensions or emulsions and sterile powders for reconstitution into sterile injectable solutions or dispersions.

Solid dosage forms for oral administration include capsules, tablets, pills, powders, and granules. In such solid dosage forms, one or more compounds of the invention is mixed with at least one inert pharmaceutically acceptable carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using lactose or milk sugar as well as high molecular weight polyethylene glycols. The solid dosage forms of tablets, dragees, capsules, pills, and granules can be prepared with coatings and shells such as enteric coatings and other coatings well-known in the pharmaceutical formulating art. They can optionally contain opacifying agents and can also be of a composition that they release the active ingredient(s) only, or preferentially, in a certain part of the intestinal tract in a delayed manner. Examples of materials useful for delaying release of the active agent can include polymeric substances and waxes.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. A desired compound of the invention is admixed under sterile conditions with a pharmaceutically acceptable carrier and any needed preservatives or buffers as may be required. Ophthalmic formulation, eardrops, eye ointments, powders and solutions are also contemplated as being within the scope of this invention.

The compounds of the invention can be used in the form of pharmaceutically acceptable salts, esters, or amides derived from inorganic or organic acids. The term “pharmaceutically acceptable salts, esters and amides,” as used herein, include salts, zwitterions, esters and amides of compounds of formula (I) which are, within the scope of sound medical judgment, suitable for use in contact with the tissues of humans and lower animals without undue toxicity, irritation, allergic response, and the like, are commensurate with a reasonable benefit/risk ratio, and are effective for their intended use.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well-known in the art. The salts can be prepared in situ during the final isolation and purification of the compounds of the invention or separately by reacting a free base function with a suitable organic acid.

Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate.

Also, the basic nitrogen-containing groups can be quaternized with such agents as lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; arylalkyl halides such as benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylamine, dimethylamine, trimethylamine, triethylamine, diethylamine, ethylamine and the such as. Other representative organic amines useful for the formation of base addition salts include ethylenediamine, ethanolamine, diethanolamine, piperidine, and piperazine.

The term “pharmaceutically acceptable ester,” as used herein, refers to esters of compounds of the invention which hydrolyze in vivo and include those that break down readily in the human body to leave the parent compound or a salt thereof. Examples of pharmaceutically acceptable, non-toxic esters of the invention include C₁-to-C₆ alkyl esters and C₅-to-C₇ cycloalkyl esters, although C₁-to-C₄ alkyl esters are preferred. Esters of the compounds of formula (I) can be prepared according to conventional methods. Pharmaceutically acceptable esters can be appended onto hydroxy groups by reaction of the compound that contains the hydroxy group with acid and an alkylcarboxylic acid such as acetic acid, or with acid and an arylcarboxylic acid such as benzoic acid. In the case of compounds containing carboxylic acid groups, the pharmaceutically acceptable esters are prepared from compounds containing the carboxylic acid groups by reaction of the compound with base such as triethylamine and an alkyl halide, alkyl trifilate, for example with methyl iodide, benzyl iodide, cyclopentyl iodide. They also can be prepared by reaction of the compound with an acid such as hydrochloric acid and an alkylcarboxylic acid such as acetic acid, or with acid and an arylcarboxylic acid such as benzoic acid.

The term “pharmaceutically acceptable amide,” as used herein, refers to non-toxic amides of the invention derived from ammonia, primary C₁-to-C₆ alkyl amines and secondary C₁-to-C₆ dialkyl amines. In the case of secondary amines, the amine can also be in the form of a 5- or 6-membered heterocycle containing one nitrogen atom. Amides derived from ammonia, C₁-to-C₃ alkyl primary amides and C₁-to-C₂ dialkyl secondary amides are preferred. Amides of the compounds of formula (I) can be prepared according to conventional methods. Pharmaceutically acceptable amides can be prepared from compounds containing primary or secondary amine groups by reaction of the compound that contains the amino group with an alkyl anhydride, aryl anhydride, acyl halide, or aroyl halide. In the case of compounds containing carboxylic acid groups, the pharmaceutically acceptable esters are prepared from compounds containing the carboxylic acid groups by reaction of the compound with base such as triethylamine, a dehydrating agent such as dicyclohexyl carbodiimide or carbonyl diimidazole, and an alkyl amine, dialkylamine, for example with methylamine, diethylamine, piperidine. They also can be prepared by reaction of the compound with an acid such as sulfuric acid and an alkylcarboxylic acid such as acetic acid, or with acid and an arylcarboxylic acid such as benzoic acid under dehydrating conditions as with molecular sieves added. The composition can contain a compound of the invention in the form of a pharmaceutically acceptable prodrug.

The term “pharmaceutically acceptable prodrug” or “prodrug,” as used herein, represents those prodrugs of the compounds of the invention which are, within the scope of sound medical judgment, suitable for use in contact with the tissues of humans and lower animals without undue toxicity, irritation, allergic response, and the like, commensurate with a reasonable benefit/risk ratio, and effective for their intended use. Prodrugs of the invention can be rapidly transformed in vivo to a parent compound of formula (I), for example, by hydrolysis in blood. A thorough discussion is provided in T. Higuchi and V. Stella, Prodrugs as Novel Delivery Systems, V. 14 of the A.C.S. Symposium Series, and in Edward B. Roche, ed., Bioreversible Carriers in Drug Design, American Pharmaceutical Association and Pergamon Press (1987).

The invention contemplates pharmaceutically active compounds either chemically synthesized or formed by in vivo biotransformation to compounds of formula (I).

### c) PREPARATION OF THE COMPOUNDS OF THE INVENTION

The compounds and processes of the present invention will be better understood in connection with the following synthetic schemes, which illustrate the methods by which the compounds of the invention may be prepared. Starting materials can be obtained from commercial sources or prepared by well-established literature methods known to those of ordinary skill in the art.

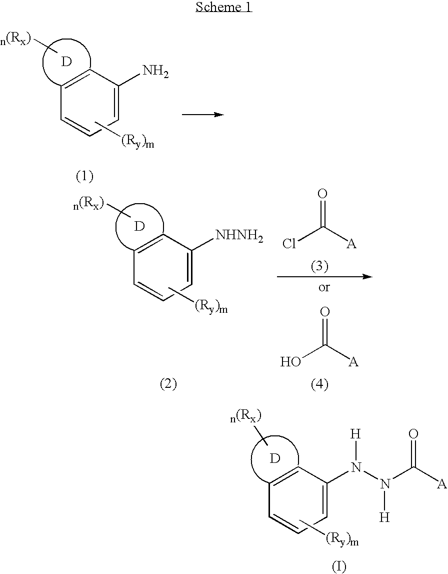

Compounds of formula (I) wherein A, Rₓ, R_{y}, m and n are as defined in formula (I) can be prepared from amines of formula (1), either purchased or synthesized by methodologies known to one skilled in the art, as shown in Scheme 1.

Some reaction conditions for the synthesis of amines of formula (1) can be found in the following references: J. Heterocyclic Chem. 1975, 12, p. 877; and Heterocycles, 1997, 45, p. 234.

Amines of formula (1) can be converted to hydrazines of formula (2) by (a) treating the amine with concentrated hydrochloric acid and aqueous sodium nitrite; and (b) treating the product of step (a) with tin(II) chloride. The reactions of step (a) and (b) are generally conducted at the temperature of about 0° C. to about room temperature.

Hydrazines of formula (2) can be converted to compounds of formula (I) by reacting with acid chlorides of formula (3), purchased or prepared from the corresponding acids, in the presence of a base such as, but not limited to, triethylamine. The reaction can be performed at a temperature from about 0° C. to about room temperature, in a suitable solvent such as, but not limited to, dichloromethane, tetrahydrofuran, ethyl acetate, toluene, acetonitrile, ether and the like, for a period of about 1 hour to about 24 hours.

Alternatively, hydrazines of formula (2) can be treated with acids of formula (4) in the presence of a coupling agent such as, but not limited to, O-benzotriazol-1-yl-N,N,N′,N′-tetramethyluronium tetrafluoroborate (TBTU), and a base such as, but not limited to, trimethyl amine. The reaction is generally performed in a suitable solvent such as, but not limited to, acetonitrile, N,N-dimethylformamide and the like.

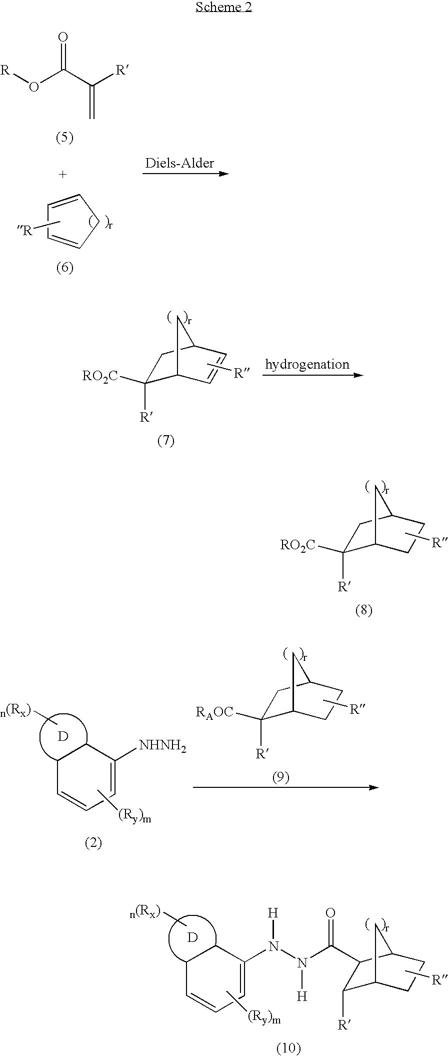

Compounds of formula (10) wherein r is 1 or 2, Rₓ, R_{y}, m, and n are as defined in formula (I), and R′ and R″ are hydrogen or are as defined in the substituents of R₁ of formula (I).

Dienes of formula (6) can be reacted with dienophiles of formula (5) wherein R is hydrogen, alkyl, alkoxyalkyl, or alkoxy. The reaction is generally conducted in a solvent such as, but not limited to, benzene, toluene, or xylene at a temperature from about 0° C. to about 150° C. The Diels-Alder Reactions may be catalyzed by a variety of Lewis acids, includng AlCl3 and optically enriched adducts may be obtained using a variety of chiral Lewis acids in solvents such as ether or tetrahydrofuran at temperatures ranging from −78° C. to 50° C. Hydrogenation of the alkenes of formula (7) provides the desired intermediate of formula (8).

Compounds of formula (8) wherein R is alkyl can be converted to compounds of formula (9) wherein R_{A} is —OH by base hydrolysis using reaction conditions that are well known in the art. Compounds of formula (8) wherein R_{A} is hydrogen can be converted to acid chlorides of formula (9) wherein R_{A} is Cl in the presence of catalytic amount of N,N dimethylformamide, thionyl chloride and a base.

Compounds of formula (9) wherein R_{A} is —OH or —Cl can be reacted with hydrazines of formula (2) using the reaction conditions as described in Scheme 1 for the conversion of hydrazines of formula (2) to compounds of formula (1).

### d) REFERENCE EXAMPLES

The following Examples are intended as an illustration of and not a limitation upon the scope of the invention as defined in the appended claims.

### EXAMPLE 1

### N′-(2-methylphenyl)adamantane-1-carbohydrazide

To an oven-dried, 25-mL, round-bottomed flask containing a magnetic stir bar was added the hydrochloride salt of o-tolylhydrazine (174 mg, 1.10 mmol). The flask was sealed with a septum and purged with nitrogen atmosphere. Anhydrous tetrahydrofuran (9 mL) was added via syringe to form a white slurry. Triethylamine (419 μL, 3.00 mmol) was added via syringe. A solution of 1-chlorocarbonyl adamantane (199 mg, 1.00 mmol) in anhydrous tetrahydrofuran (1 mL) was added. The white slurry was stirred at room temperature for 2 hours and then monitored by LC-MS (Hewlett-Packard 1100 HPLC with a Finnigan Navigator MS; C18 reverse phase column; 10-100% acetonitrile: 10 mM ammonium acetate gradient; APCI positivie ionization) Water (10 mL) was added to quench and the reaction mixture was transferred to a separatory funnel. The mixture was extracted with dichloromethane (3×8 mL). The combined organic extracts were dried over magnesium sulfate, filtered, and concentrated by rotary evaporator to give an off-white solid. The product was re-crystallized from ethyl acetate/hexanes to give 220 mg (77%) of a white powder. MS (ESI⁺) m/z 285.1 (M+H)⁺; ¹HNMR (CDCl₃) δ 1.72-1.81 (m, 6H), 1.95-1.96 (m, 6H), 2.09 (br s, 3H), 2.26 (s, 3H), 3.60 (br s, 1H), 6.78-6.86 (m, 2H), 7.06-7.13 (m, 2H), 7.38 (br s, 1H). Anal. Calc'd for C₁₈H₂₄N₂O: C, 76.02; H, 8.51; N, 9.85. Found: C, 75.72; H, 8.73; N, 9.75.

### EXAMPLE 2

### N′-(2-methylphenyl)-4-pentylbicyclo[2.2.2]octane-1-carbohydrazide

The title compound was prepared using the procedure as described in Example 1, substituting the acid chloride of 4-pentyl-bicyclo[2.2.2]octane-1-carboxylic acid for 1-chlorocarbonyl adamantane. MS (ESI⁺) m/z 329.1 (M+H)⁺; ¹HNMR (CDCl₃) δ 0.88 (t, J=7.0 Hz, 3H), 1.07-1.30 (m, 8H), 1.41-1.47 (m, 6H), 1.79-1.85 (m, 6H), 2.93 (s, 3H), 6.77 (d, J=7.8 Hz, 1H), 6.80-6.85 (m, 1H), 7.05-7.12 (m, 2H), 7.30 (br s, 1H). Anal. calc'd for C₂₁H₃₂N₂O: C, 76.78; H, 9.82; N, 8.53. Found: C, 76.02; H, 10.38; N, 8.58.

### EXAMPLE 3

### N′-(2-methylphenyl)-1-phenylcyclopentanecarbohydrazide

The title compound was prepared using the procedure as described in Example 1, substituting the acid chloride of 1-phenyl-cyclopentanecarboxylic acid for 1-chlorocarbonyl adamantane. MS (ESI⁺) m/z 295.1 (M+H)⁺; ¹HNMR (CDCl₃) δ 1.70-1.78 (m, 2H), 1.80-1.90 (m, 2H), 2.07-2.16 (m, 2H), 2.19 (s, 3H), 2.51-2.59 (m, 2H), 6.47 (d, J=7.8 Hz, 1H), 6.79 (dd, J=7.0, 7.0 Hz, 1H), 6.86 (br s, 1H), 7.00-7.04 (m, 2H), 7.29-7.34 (, 1H), 7.38-7.46 (m, 4H).

### EXAMPLE 4

### N′-(2-methylphenyl)-1-phenylcyclopentanecarbohydrazide

The title compound was prepared using the procedure as described in Example 1, substituting the acid chloride of 3-noradamantanecarboxylic acid for 1-chlorocarbonyl adamantane. MS (ESI⁺) m/z 271.0 (M+H)⁺; ¹HNMR (CDCl₃) δ 1.63-1.70 (m, 4H), 1.81-1.91 (m, 4H), 2.04-2.09 (m, 2H), 2.27 (s, 3H), 2.37 (br s, 2H), 2.76 (t, J=6.8 Hz, 1H), 6.80-6.86 (m, 2H), 7.07-7.14 (m, 2H), 7.33 (br s, 1H). Anal. calc'd for C₁₇H₂₂N₂O: C, 75.52; H, 8.20; N, 10.36. Found: C, 74.84; H, 8.28; N, 10.32.

### EXAMPLE 5

### N′-(2-methylphenyl)butanohydrazide

A vial containing a stir bar was charged polymer-supported-carbodiimide resin (3.00 equivalents). To the vessel was added the butyric acid (1.25 equivalents), hydroxybenzotriazole (1.00 equivalent) and a solution of diisopropylethylamine (3.00 equivalents) with the hydrochloride salt of o-tolylhydrazine (1.00 equivalent) in dimethylacetamide. The reaction vessel was sealed and heated at 100° C. for 420 seconds in a microwave reactor. After cooling, the reaction mixture was transferred to a prepacked column of Si-Carbonate (>4 equivalents of functionalized reagent), which had been previously conditioned with methanol. The reaction products were collected and concentrated to dryness. The residues were dissolved in 1:1 dimethylsulfoxide:methanol and purified by reverse phase HPLC (Waters Symmetry C8 column using a gradient of 10% to 100% acetonitrile: 10 mM ammonium acetate) to afford the title compound. MS (ESI⁺) m/z 192.9 (M+H)⁺; ¹HNMR (DMSO-d₆/D₂O) δ 0.92 (t, J=7.5 Hz, 3H), 1.55-1.63 (m, 2H), 2.15 (s, 3H), 2.18 (t, J=7.3 Hz, 2H), 6.64 (d, J=8.1 H, 1H), 6.69 (dd, J=6.9, 6.9 Hz, 1H), 7.01-7.04 (m, 2H).

### EXAMPLE 6

### N′-(2-methylphenyl)pentanohydrazide

The title compound was prepared using the procedure as described in Example 5, substituting pentanoic acid for butyric acid. MS (ESI⁺) m/z 207.0 (M+H)⁺; ¹HNMR (DMSO-d₆/D₂O) δ 0.90 (t, J=7.3 Hz, 3H), 1.29-1.36 (m, 2H), 1.52-1.58 (m, 2H), 2.14 (s, 3H), 2.20 (t, J=7.5 Hz, 2H), 6.63 (d, J=7.8 Hz, 1H), 6.69 (dd, J=6.9, 6.9 Hz, 1H), 7.01-7.04 (m, 2H).

### EXAMPLE 7

### 3-methyl-N′-(2-methylphenyl)butanohydrazide

The title compound was prepared using the procedure as described in Example 5, substituting 3-methylbutyric acid for butyric acid. MS (ESI⁺) m/z 206.9 (M+H)⁺; ¹HNMR (DMSO-d₆/D₂O) δ 0.94 (d, J=6.2 Hz, 6H), 2.02-2.06 (m, 1H), 2.07 (d, J=7.8 Hz, 2H), 2.15 (s, 3H), 6.66 (d, J=8.1 Hz, 1H), 6.69 (dd, J=7.3, 7.2 Hz, 1H), 7.01-7.04 (m, 2H).

### EXAMPLE 8

### 2,2-dimethyl-N′-(2-methylphenyl)propanohydrazide

The title compound was prepared using the procedure as described in Example 5, substituting 2,2-dimethylpropionic acid for butyric acid. MS (ESI⁺) m/z 206.9 (M+H)⁺; ¹HNMR (DMSO-d₆/D₂O) δ 1.20 (s, 9H), 2.16 (s, 3H), 6.62 (d, J=7.8 Hz, 1H), 6.69 (dd, J=7.3, 7.3 Hz, 1H), 7.01-7.04 (m, 2H).

### EXAMPLE 9

### N′-(2-methylphenyl)hexanohydrazide

The title compound was prepared using the procedure as described in Example 5, substituting hexanoic acid for butyric acid. MS (ESI⁺) m/z 221.0 (M+H)⁺; ¹HNMR (DMSO-d₆/D₂O) δ 0.888 (t, J=7.0 Hz, 3H), 1.27-1.33 (m, 2H), 1.54-1.60 (m, 2H), 2.14 (s, 3H), 2.19 (t, J=7.5 Hz, 2H), 6.64 (d, J=8.1 HZ, 1H), 6.68 (dd, J=7.3, 7.3 Hz, 1H), 7.01-7.04 (m, 2H).

### EXAMPLE 10

### 2-methyl-N′-(2-methylphenyl)pentanohydrazide

The title compound was prepared using the procedure as described in Example 5, substituting 2-methylpentanoic acid for butyric acid. MS (ESI⁺) m/z 220.9 (M+H)⁺; ¹HNMR (DMSO-d₆/D₂O) δ 0.89 (t, J=7.2 (Hz, 1H), 1.07 (d, J=6.9 Hz, 1H), 1.25-1.34 (m, 3H), 1.52-1.58 (m, 1H), 2.15 (s, 3H), 2.38-2.42 (m, 1H), 6.64 (d, J=7.8 Hz, 1H), 6.69 (dd, J=7.3, 7.3 Hz, 1H), 7.01-7.04 (m, 2H).

### EXAMPLE 11

### 3-methyl-N′-(2-methylphenyl)pentanohydrazide

The title compound was prepared using the procedure as described in Example 5, substituting 3-methylpentanoic acid for butyric acid. MS (ESI⁺) m/z 221.0 (M+H)⁺; ¹HNMR (DMSO-d₆/D₂O) δ 0.88 (t, J=7.5 Hz, 3H), 0.91 (D, J=6.6 Hz, 3H), 1.16-1.25 (m, 1H), 1.32-1.39 (m, 1H), 1.79-1.86 (m, 1H), 1.98-2.03 (m, 1H), 2.15 (s, 3H), 2.17-2.21 (m, 1H), 6.65 (d, J=7.5 Hz, 1H), 6.69 (dd, J=7.3, 7.2 Hz, 1H), 7.01-7.04 (m, 2H).

### EXAMPLE 12

### 4-methyl-N′-(2-methylphenyl)pentanohydrazide

The title compound was prepared using the procedure as described in Example 5, substituting 4-methylpentanoic acid for butyric acid. MS (ESI⁺) m/z 221.0 (M+H)⁺; ¹HNMR (DMSO-d₆/D₂O) δ 0.90 (d, J=6.6 Hz, 6H), 1.44-1.48 (m, 2H), 1.56 (sept, J=6.9 Hz, 1H), 2.14 (s, 3H), 2.20 (t, J=7.8 Hz, 2H), 2.53-2.54 (m, 1H), 6.63 (D, J=7.8 Hz, 1H), 6.68 (dd, J=7.3, 7.3 Hz, 1H), 7.01-7.04 (m, 2H).

### EXAMPLE 13

### 2,2-dimethyl-N′-(2-methylphenyl)butanohydrazide

The title compound was prepared using the procedure as described in Example 5, substituting 2,2-dimethylbutyric acid for butyric acid. MS (ESI⁺) m/z 221.0 (M+H)⁺; ¹HNMR (DMSO-d₆/D₂O) δ 0.82 (t, J=7.5 Hz, 3H)1.15 (s, 6H), 1.56 (q, J=7.5 Hz, 2H), 2.17 (s, 3H), 6.65(d, J=8.4 Hz, 1H), 6.69 (dd, J=7.3, 7.3 Hz, 1H), 7.01-7.04 (m, 2H).

### EXAMPLE 14

### 3,3-dimethyl-N′-(2-methylphenyl)butanohydrazide

The title compound was prepared using the procedure as described in Example 5, substituting 3,3-dimethylbutyric acid for butyric acid. MS (ESI⁺) m/z 221.0 (M+H)⁺; ¹HNMR (DMSO-d₆/D₂O) δ 1.02 (s, 9H), 2.08 (s, 2H), 2.15 (s, 3H), 6.68-6.70 (m, 2H), 7.01-7.04 (m, 2H).

### EXAMPLE 15

### 2-ethyl-N′-(2-methylphenyl)butanohydrazide

The title compound was prepared using the procedure as described in Example 5, substituting 2-ethylbutyric acid for butyric acid. MS (ESI⁺) m/z 221.0 (M+H)⁺; ¹HNMR (DMSO-d₆/D₂O) δ 0.88 (t, J=7.5 Hz, 6H), 1.39-1.46 (m, 2H), 1.48-1.56 (m, 2H), 2.08-2.14 (m, 1H), 2.16 (s, 3H), 6.68-6.70 (m, 2H), 7.01-7.04 (m, 2H).

### EXAMPLE 16

### N′-(2-methylphenyl)heptanohydrazide

The title compound was prepared using the procedure as described in Example 5, substituting heptanoic acid for butyric acid. MS (ESI⁺) m/z 235.0 (M+H)⁺; ¹HNMR (DMSO-d₆/D₂O) δ 0.88 (t, J=7.6 Hz, 3H), 1.18-1.33 (m 6H), 1.53-1.59 (m, 2H), 2.14 (S, 3H), 2.19 (t, J=7.3 Hz, 2H), 6.64 (d, J=7.8 Hz, 1H), 6.68 (dd, J=7.3, 7.3 Hz, 1H), 7.00-7.03 (m, 2H).

### EXAMPLE 17

### 2-cyclopentyl-N′-(2-methylphenyl)acetohydrazide

The title compound was prepared using the procedure as described in Example 5, substituting cyclopentylacetic acid for butyric acid. MS (ESI⁺) m/z 233.0 (M+H)⁺; ¹HNMR (DMSO-d₆/D₂O) δ 1.16-1.22 (m, 2H), 1.49-1.55 (m, 2H), 1.58-1.64 (m, 2H), 1.72-1.78 (m, 2H), 2.15 (s, 3H),2.16-2.19 (m, 1H), 2.20 (d, J=6.8 Hz, 2H), 6.65 (d, J=7.8 Hz, 1H), 6.88 (dd, J=7.3, 7.3 Hz, 1H), 7.01-7.04 (m, 2H).

### EXAMPLE 18

### 1-methyl-N′-(2-methylphenyl)cyclohexanecarbohydrazide

The title compound was prepared using the procedure as described in Example 5, substituting 1-methylcyclohexanecarboxylic acid for butyric acid. MS (ESI⁺) m/z 247.0 (M+H)⁺; ¹HNMR (DMSO-d₆/D₂O) δ 1.17 (s, 3H), 1.24-1.36 (m, 2H), 1.36-1.48 (m, 2H), 1.48-1.54 (m, 2H), 1.99-2.02 (m, 2H), 2.17 (s, 3H), 6.66 (d, J=8.4 Hz, 1H), 6.68 (dd, J=7.3, 7.3 Hz, 1H), 7.01-7.04 (m, 2H).

### EXAMPLE 19

### 2-cyclohexyl-N′-(2-methylphenyl)acetohydrazide

The title compound was prepared using the procedure as described in Example 5, substituting cyclohexylacetic acid for butyric acid. MS (ESI⁺) m/z 247.0 (M+H)⁺; ¹HNMR (DMSO-d₆/D₂O) δ 0.93-1.0 (m, 2H), 1.12-1.25 (m, 3H), 1.60-1.74 (m, 6H), 2.08 (d, J=6.9 Hz, 2H), 2.14 (s, 3H), 6.64 (d, J=7.5 Hz, 1H), 6.68 (dd, J=7.3, 7.3 Hz, 1H), 7.01-7.04 (m, 2H).

### EXAMPLE 20

### 2-(1-adamantyl)-N′-(2-methylphenyl)acetohydrazide

The title compound was prepared using the procedure as described in Example 5, substituting adamantanyl-1-ylacetic acid for butyric acid. MS (ESI⁺) m/z 299.0 (M+H)⁺; ¹HNMR (DMSO-d₆/D₂O) δ 1.58-1.69 (m, 12H), 1.93 (br s, 3H),1.96 (s, 2H), 2.15 (s, 3H), 6.61 (d, J=7.5 HZ, 1H), 6.68 (dd, J=7.3, 7.3 Hz, 1H), 7.00-7.03 (m, 2H).

### EXAMPLE 21

### 3-ethoxy-N′-(2-methylphenyl)propanohydrazide

The title compound was prepared using the procedure as described in Example 5, substituting 3-ethoxypropionic acid for butyric acid. MS (ESI⁺) m/z 223.0 (M+H)⁺; ¹HNMR (DMSO-d₆/D₂O) δ 1.26 (t, J=7.0 Hz, 3H), 2.26 (s, 3H), 2.54 (t, J=6.2 Hz, 2H), 3.57 (q, J=7.0 Hz, 2H), 3.76 (q, J=6.2 Hz, 2H), 6.79-6.85 (m, 2H), 7.07-7.13 (m, 2H).

### EXAMPLE 22

### N′-(2-methylphenyl)-3-phenylpropanohydrazide

The title compound was prepared using the procedure as described in Example 5, substituting 3-phenylpropionic acid for butyric acid. MS (ESI⁺) m/z 254.9 (M+H)⁺; ¹HNMR (DMSO-d₆/D₂O) δ 2.12 (s, 3H), 2.53 (t, J=7.5 Hz, 2H), 2.89 (t, J=7.5 Hz, 2H), 6.31 (d, J=7.8 Hz, 1H), 6.66 (t, J=6.9 Hz, 1H), 6.91 (dd, J=7.8, 7.3 Hz, 1H), 6.99 (d, J=7.2 Hz, 1H), 7.22-7.36 (m, 3H), 7.30-7.33 (m, 2H).

### EXAMPLE 23

### 4-methoxy-N′-(2-methylphenyl)cyclohexanecarbohydrazide

The title compound was prepared using the procedure as described in Example 5, substituting 4-methoxycyclohexanecarboxylic acid for butyric acid. MS (ESI⁺) m/z 263.0 (M+H)⁺.

### EXAMPLE 24

### N′-(2-methylphenyl)-1-phenylcyclopropanecarbohydrazide

The title compound was prepared using the procedure as described in Example 5, substituting 1-phenylcyclopropanecarboxylic acid for butyric acid. MS (ESI⁺) m/z 267.0 (M+H)⁺; ¹HNMR (DMSO-d₆/D₂O) δ 1.06-1.09 (m, 2H), 1.39-1.41 (m, 2H), 2.10 (s, 3H), 6.52 (d, J=7.8 Hz, 1H), 6.67 (dd, J=7.2, 6.9 Hz, 1H), 6.99-7.02 (m, 2H), 7.30-7.33 (m, 1H), 7.39 (dd, J=7.5 Hz, 2H), 7.44-7.46 (m, 2H).

### EXAMPLE 25

### (2S)-N′-(2-methylphenyl)-2-phenylbutanohydrazide

The title compound was prepared using the procedure as described in Example 5, substituting (S)-2-phenylbutyric acid for butyric acid. MS (ESI⁺) m/z 268.9 (M+H)⁺; ¹HNMR (DMSO-d₆/D₂O) δ 0.89 (t, J=7.3 Hz, 3H), 1.68-1.74 (m, 1H), 1.97-2.06 (m, 1H), 2.11 (s, 3H), 3.48 (dd, J=7.2, 6.6 Hz, 1H), 6.44 (d, J=7.2 Hz, 1H), 6.64 (dd, J=7.3, 7.3 Hz, 1H), 6.89 (dd, J=7.2, 7.2 Hz, 1H), 6.99 (d, J=7.5 Hz, 1H), 7.25-7.28 (m, 1H), 7.32-7.35 (m, 2H), 7.37-7.39 (m, 2H).

### EXAMPLE 26

### N′-(2-methylphenyl)-4-phenylbutanohydrazide

The title compound was prepared using the procedure as described in Example 5, substituting 4-phenylbutyric acid for butyric acid. MS (ESI⁺) m/z 269.1 (M+H)⁺. ¹HNMR (DMSO-d₆/D₂O) δ 1.84-1.90 (m, 2H),2.15 (s, 3H), 2.23 (t, J=7.5 Hz, 2H), 2.62 (t, J=7.5 Hz, 2H), 6.64 (d, J=7.2 Hz, 1H), 6.69 (dd, J=6.9, 6.9 Hz, 1H), 7.01-7.04 (m, 2H), 7.19-7.22 (m, 3H), 7.29-7.32 (m, 2H).

### EXAMPLE 27

### (2R)-2-methoxy-N′-(2-methylphenyl)-2-phenylacetohydrazide

The title compound was prepared using the procedure as described in Example 5, substituting (R)-1-methoxyphenylacetic acid for butyric acid. MS (ESI⁺) m/z 270.9 (M+H)⁺; ¹HNMR (DMSO-d₆/D₂O) δ 2.12 (s, 3H), 3.37 (s, 3H), 4.81 (s, 1H), 6.47 (d, J=7.8 Hz, 1H), 6.66 (dd, J=6.9, 6.9 Hz, 1H), 6.92 (dd, J=7.8, 7.8 Hz, 1H), 6.99 (d, J=7.2 Hz, 1H), 7.34-7.42 (m, 3H), 7.48 (d, J=7.2 Hz, 2H).

### EXAMPLE 28

### (2S)-2-methoxy-N′-(2-methylphenyl)-2-phenylacetohydrazide

The title compound was prepared using the procedure as described in Example 5, substituting (S)-1-methoxyphenylacetic acid for butyric acid. MS (ESI⁺) m/z 271.0 (M+H)⁺; ¹HNMR (DMSO-d₆/D₂O) δ 2.12 (s, 3H), 3.37 (s, 3H), 4.81 (s, 1H), 6.47 (d, J=7.8 Hz, 1H), 6.66 (dd, J=6.9, 6.9 Hz, 1H), 6.92 (dd, J=7.8, 7.8 Hz, 1H), 6.99 (d, J=7.2 Hz, 1H), 7.34-7.42 (m, 3H), 7.48 (d, J=7.2 Hz, 2H).

### EXAMPLE 29

### N′-(2-methylphenyl)-3-phenoxypropanohydrazide

The title compound was prepared using the procedure as described in Example 5, substituting 3-phenoxypropionic acid for butyric acid. MS (ESI⁺) m/z 271.0 (M+H)⁺; ¹HNMR (DMSO-d₆/D₂O) δ 2.15 (s, 3H), 2.67 (t, J=5.8 Hz, 2H), 4.24 (t, J=5.9 Hz, 2H), 6.68 (dd, J=7.2, 7.3 Hz, 1H), 6.75 (d, J=7.2 Hz, 1H), 6.95-7.02 (m, 5H), 7.32 (dd, J=7.2 Hz, 2H).

### EXAMPLE 30

### N′-(2-methylphenyl)-2-((furan-2-yl)carbonylamino)acetohydrazide

The title compound was prepared using the procedure as described in Example 5, substituting [(furan-2-carbonyl)-amino]-acetic acid for butyric acid. MS (ESI⁺) m/z 273.9 (M+H)⁺; ¹HNMR (DMSO-d₆/D₂O) δ 2.14 (s, 3H), 3.96 (br s, 2H), 6.64-6.66 (m, 2H), 6.69 (dd, J=7.3 Hz, 1H), 6.74 (d, J=7.5 Hz, 1H), 7.00-7.02 (m, 2H), 7.15 (d, J=4.4 Hz, 1H), 7.82 (br s, 1H).

### EXAMPLE 31

### N′-(2-methylphenyl)-2-(pyrimidin-2-ylthio)acetohydrazide

The title compound was prepared using the procedure as described in Example 5, substituting (pyrimidin-2-ylsulfanyl)-acetic acid for butyric acid. MS (ESI⁺) m/z 274.9 (M+H)⁺; ¹HNMR (DMSO-d₆/D₂O) δ 2.13 (s, 3H), 3.97 (s, 2H), 6.68 (dd, J=7.3, 7.2 Hz, 1H), 6.74 (d, J=7.5 Hz, 1H), 6.99-7.02 (m, 2H), 7.26 (t, J=5.0 Hz, 1H), 8.66 (d, J=5.0 Hz, 2H).

### EXAMPLE 32

### N′-(2-methylphenyl)-4-thien-2-ylbutanohydrazide

The title compound was prepared using the procedure as described in Example 5, substituting 4-thiophen-2-ylbutyric acid for butyric acid. MS (ESI⁺) m/z 274.9 (M+H)⁺; ¹HNMR (DMSO-d₆/D₂O) δ 2.15 (s, 3H), 1.88-1.94 (m, 2H), 2.27 (t, J=7.3 Hz, 2H), 2.84 (t, J=7.5 Hz, 2H), 6.64 (D, J=7.8 Hz, 1H), 6.69 (dd, J=7.0, 6.9 Hz, 1H), 6.87-6.88 (m, 1H), 6.97 (dd, J=5.0, 3.4 Hz, 1H), 7.01-7.04 (m, 2H), 7.31 (dd, J=5.0, 1.2 Hz, 1H).

### EXAMPLE 33

### 2-(3,5-difluorophenyl)-N′-(2-methylphenyl)acetohydrazide

The title compound was prepared using the procedure as described in Example 5, substituting (3,5-difluorophenyl)acetic acid for butyric acid. MS (ESI⁺) m/z 276.9 (M+H)⁺; ¹HNMR (DMSO-d₆/D₂O) δ 2.13 (S, 3H), 3.59 (s, 2H), 6.59 (d, J=7.8 Hz, 1H), 6.69 (dd, J=7.0, 6.9 Hz, 1H), 6.97-7.02 (m, 2H), 7.03-7.12 (m, 3H).

### EXAMPLE 34

### N′-(2-methylphenyl)-2-((2S)-acetylamino)-4-methylpentanohydrazide

The title compound was prepared using the procedure as described in Example 5, substituting (S)-2-acetylamino-4-methyl-pentanoic acid for butyric acid. MS (ESI⁺) m/z 278.0 (M+H)⁺; ¹HNMR (DMSO-d₆/D₂O) δ 0.88 (d, J=6.6 Hz, 3H), 0.94 (d, J=6.9 Hz, 3H), 1.48-1.54 (m, 2H), 1.62-1.66 (m, 1H), 1.88 (s, 3H), 2.14 (s, 3H), 4.38 (dd, J=9.0, 6.3 Hz, 1H), 6.66-6.70 (m, 2H), 7.00-7.02 (m, 2H).

### EXAMPLE 35

### N′-(2-methylphenyl)-4-oxo-4-phenyl-3-azabutanohydrazide

The title compound was prepared using the procedure as described in Example 5, substituting benzoylaminoacetic acid for butyric acid. MS (ESI⁺) m/z 283.9 (M+H)⁺; ¹HNMR (DMSO-d₆/D₂O) δ 2.15 (s, 3H), 4.01 (s, 2H), 6.70 (dd, J=7.3, 7.2 Hz, 1H), 6.76 (d, J=8.1 Hz, 1H), 7.00-7.04 (m, 2H), 7.48-7.51 (m, 3H), 7.55-7.58 (m, 1H), 7.89 (d, J=8.2 Hz, 2H).

### EXAMPLE 36

### 3-(3-methoxyphenyl)-N′-(2-methylphenyl)propanohydrazide

The title compound was prepared using the procedure as described in Example 5, substituting 3-(3-methoxyphenyl)propionic acid for butyric acid. MS (ESI⁺) m/z 284.9 (M+H)⁺.

### EXAMPLE 37

### 3-(4-methoxyphenyl)-N′-(2-methylphenyl)propanohydrazide

The title compound was prepared using the procedure as described in Example 5, substituting 3-(4-methoxyphenyl)propionic acid for butyric acid. MS (ESI⁺) m/z 284.9 (M+H)⁺; ¹HNMR (DMSO-d₆/D₂O) δ 2.12 (s, 3H), 2.48 (t, J=7.4 Hz, 2H), 2.83 (t, J=7.3 Hz, 2H), 3.74 (s, 3H), 6.29 (d, J=7.8 Hz, 1H), 6.65 (dd, J=6.9, 6.9 Hz, 1H), 6.86-6.91 (m, 3H), 6.98 (D, J=7.2 HZ, 1H), 7.16 (d, J=8.7 Hz, 2H).

### EXAMPLE 38

### (2R)-2-hydroxy-N′-(2-methylphenyl)-4-phenylbutanohydrazide

The title compound was prepared using the procedure as described in Example 5, substituting (R)-2-hydroxy-4-phenylbutyric acid for butyric acid. MS (ESI⁺) m/z 284.9 (M+H)⁺.

### EXAMPLE 39

### N′-(2-methylphenyl)-4-phenoxybutanohydrazide

The title compound was prepared using the procedure as described in Example 5, substituting 4-phenoxybutyric acid for butyric acid. MS (ESI⁺) m/z 285.0 (M+H)⁺. ¹HNMR (DMSO-d₆/D₂O) δ 2.01 (t, J=7.0 Hz, 2H), 2.14 (S, 3H), 2.39 (t, J=7.3 Hz, 2H), 4.00, (t, J=7.3 Hz, 2H), 6.61 (d, J=7.8 Hz, 1H), 6.67 (dd, J=7.5, 7.5 Hz, 1H), 6.92-6.96 (m, 4H), 7.01 (d, J=7.5 Hz, 1H), 7.29-7.32 (m, 2H).

### EXAMPLE 40

### N′-(2-methylphenyl)-4-oxo-4-thien-2-ylbutanohydrazide

The title compound was prepared using the procedure as described in Example 5, substituting 4-oxo-4-thiophen-2-yl-butyric acid for butyric acid. MS (ESI⁺) m/z 288.9 (M+H)⁺.

### EXAMPLE 41

### 3-(2-chlorophenyl)-N′-(2-methylphenyl)propanohydrazide

The title compound was prepared using the procedure as described in Example 5, substituting 3-(2-chlorophenyl)propionic acid for butyric acid. MS (ESI⁺) m/z 288.9 (M+H)⁺; ¹HNMR (DMSO-d₆/D₂O) δ 2.13 (s, 3H), 2.55 (t, J=7.5 Hz, 2H), 3.00 (t, J=7.5 Hz, 2H), 6.41 (d, J=7.8 Hz, 1H), 6.67 (dd, J=7.4, 7.3 Hz, 1H), 6.95 (dd, J=7.6, 7.5 Hz, 1H), 7.00 (d, J=7.2 Hz, 1H), 7.28-7.30 (m, 2H), 7.35-7.37 (m, 1H), 7.44-7.45 (m, 1H).

### EXAMPLE 42

### 3-(4-chlorophenyl)-N′-(2-methylphenyl)propanohydrazide

The title compound was prepared using the procedure as described in Example 5, substituting 3-(4-chlorophenyl)propionic acid for butyric acid. MS (ESI⁺) m/z 289.0 (M+H)⁺; ¹HNMR (DMSO-d₆/D₂O) δ 2.11 (s, 3H), 2.52 (t, J=7.3 Hz, 2H), 2.88 (t, J=7.3 Hz, 2H), 6.21 (d, J=8.1 Hz, 1H),6.66 (dd, J=7.4, 7.3 Hz, 1H), 6.89 (dd, J=7.6, 7.5 Hz, 1H), 6.98 (d, J=7.2 Hz, 1H), 7.27 (d, J=8.4 Hz, 2H), 7.35 (d, J=8.4 Hz, 2H).

### EXAMPLE 43

### 3-methyl-N′-(2-methylphenyl)-2-phenylpentanohydrazide

The title compound was prepared using the procedure as described in Example 5, substituting 3-methyl-2-phenylpentanoic acid for butyric acid. MS (ESI⁺) m/z 297.0 (M+H)⁺.

### EXAMPLE 44

### 5-[2-(2-methylphenyl)hydrazinol-5-oxo-N-phenylpentanamide

The title compound was prepared using the procedure as described in Example 5, substituting 4-phenylcarbamoyl-butyric acid for butyric acid. MS (ESI⁺) m/z 312.1 (M+H)⁺.

### EXAMPLE 45

### 4-(4-methoxyphenyl)-N′-(2-methylphenyl)-4-oxobutanohydrazide

The title compound was prepared using the procedure as described in Example 5, substituting 4-(4-methoxy-phenyl)-4-oxo-butyric acid for butyric acid. MS (ESI⁺) m/z 313.0 (M+H)⁺.

### EXAMPLE 46

### N′-(2-methylphenyl)-2,2-diphenylacetohydrazide

The title compound was prepared using the procedure as described in Example 5, substituting diphenylacetic acid for butyric acid. MS (ESI⁺) m/z 317.2 (M+H)⁺; ¹HNMR (DMSO-d₆/D₂O) δ 2.13 (s, 3H), 5.07 (s, 1H), 6.48 (d, J=8.12 Hz, 1H), 6.66 (dd, J=7.3, 7.2 HZ, 1H), 6.91 (dd, J=7.6, 7.5 Hz, 1H), 7.00 (d, J=7.2 Hz, 1H), 7.26-7.39 (m, 10 H).

### EXAMPLE 47

### N′-(2-methylphenyl)-3-(phenylsulfonyl)propanohydrazide

The title compound was prepared using the procedure as described in Example 5, substituting 3-benzenesulfonylpropionic acid for butyric acid. MS (ESI⁺) m/z 318.9 (M+H)⁺; ¹HNMR (DMSO-d₆/D₂O) δ 2.12 (s, 3H), 2.58 (t, J=7.5 Hz, 2H), 3.56 (t, J=7.5 Hz, 2H), 6.64 (d, J=7.2 Hz, 1H), 6.68 (dd, J=7.0, 6.9 Hz, 1H), 6.99-7.02 (m, 2H), 7.71 (d, J=7.4 Hz, 2H), 7.78-7.81 (m, 1H), 7.94 (d, J=7.2 Hz, 2H).

### EXAMPLE 48

### N′-(2-methylphenyl)-2-[4-(methylsulfonyl)phenyl]acetohydrazide

The title compound was prepared using the procedure as described in Example 5, substituting (4-methanesulfonylphenyl)-acetic acid for butyric acid. MS (ESI⁺) m/z 318.6 (M+H)⁺; ¹HNMR (DMSO-d₆/D₂O) δ 2.13 (s, 3H), 3.18 (s, 2H), 3.19 (s, 3H), 6.61 (d, J=7.8 Hz, 1H), 6.68 (dd, J=6.9, 6.9 Hz, 1H), 6.98-7.01 (m, 2H), 7.61 (d, j=8.4 Hz, 2H), 7.89 (d, J=8.4 Hz, 2H).

### EXAMPLE 49

### N′-(2-methylphenyl)-2-(3-phenoxyphenyl)acetohydrazide

The title compound was prepared using the procedure as described in Example 5, substituting (3-phenoxyphenyl)acetic acid for butyric acid. MS (ESI⁺) m/z 333.0 (M+H)⁺; ¹HNMR (DMSO-d₆/D₂O) δ 2.12 (s, 3H), 3.52 (s, 2H), 6.65 (d, j=7.8 Hz, 1H), 6.67 (dd, J=7.3, 7.2 Hz, 1H), 6.90 (dd, J=7.9, 2.0 Hz, 1H), 6.94 (dd, J=7.6, 7.6 Hz, 1H), 6.99-7.02 (m, 2H), 7.11-7.17 (m, 2H), 7.34-7.42 (m, 3H).

### EXAMPLE 50

### 4-methyl-N-{5-[2-(2-methylphenyl)hydrazino]-5-oxomethyl}benzenesulfonamide

The title compound was prepared using the procedure as described in Example 5, substituting (toluene-4-sulfonylamino)-acetic acid for butyric acid. MS (ESI⁺) m/z 333.9 (M+H)⁺; ¹HNMR (DMSO-d₆/D₂O) δ 2.12 (s, 3H), 2.39 (s, 3H), 3.53 (s, 2H), 6.55 (d, J=7.8 Hz, 1H), 6.69 (dd, J=7.3, 7.2 Hz, 1H), 6.97-7.01 (m, 2H), 7.41 (d, J=8.4 Hz, 2H), 7.73 (d, J=8.1 Hz, 2H).

### EXAMPLE 51

### 2-methyl-N′-(2-methylphenyl)propanohydrazide

The title compound was prepared using the procedure as described in Example 5, substituting isobutyric acid for butyric acid. MS (ESI⁺) m/z 192.9 (M+H)⁺; ¹HNMR (DMSO-d₆/D₂O) δ 1.09 (d, J=6.9 Hz, 6H), 2.15 (s, 3H), 2.53 (sept, J=6.9 Hz, 1H), 6.63 (d, J=7.8 Hz, 1H), 6.68 (t, J=7.3, 7.2 Hz, 1H), 7.01-7.04 (m, 2H).

### EXAMPLE 52

### N′-(2-methylphenyl)-2-(methylthio)acetohydrazide

The title compound was prepared using the procedure as described in Example 5, substituting thiomethylacetic acid for butyric acid. MS (ESI⁺) m/z 210.9 (M+H)⁺. ¹HNMR (DMSO-d₆/D₂O) δ 2.15 (s, 3H), 2.18 (s, 3H), 3.18 (s, 2H), 6.69-6.72 (m, 2H), 7.02-7.05 (m, 2H).

### EXAMPLE 53

### N′-(2-methylphenyl)tetrahydrofuran-2-carbohydrazide

The title compound was prepared using the procedure as described in Example 5, substituting tetrahydrofuran-2-carboxylic acid for butyric acid. MS (ESI⁺) m/z 221.0 (M+H)⁺. ¹HNMR (DMSO-d₆/D₂O) δ 1.86-1.93 (m, 3H), 2.15 (s, 3H), 2.12-2.22 (m, 1H), 3.78-3.82 (m, 1H), 3.95-3.99 (m, 1H), 4.35-4.37 (m, 1H), 6.60 (d, J=8.1 Hz, 1H), 6.70 (dd, J=7.2, 7.2 Hz, 1H), 7.02-7.05 (m, 2H).

### EXAMPLE 54

### N′-(2-methylphenyl)pent-4-ynohydrazide

The title compound was prepared using the procedure as described in Example 5, substituting pent-4-ynoic acid for butyric acid. MS (ESI⁺) m/z 202.9 (M+H)⁺; ¹HNMR (DMSO-d₆/D₂O) δ 2.14 (s, 3H), 2.39-2.47 (m, 4H), 6.69 (dd, J=7.2, 7.2 Hz, 1H), 6.73 (d, J=7.5 Hz, 1H), 7.01-7.04 (m, 2H).

### EXAMPLE 55

### N′-(2-methylphenyl)cyclobutanecarbohydrazide

The title compound was prepared using the procedure as described in Example 5, substituting cyclobutanecarboxylic acid for butyric acid. MS (ESI⁺) m/z 204.9 (M+H)⁺; ¹HNMR (DMSO-d₆/D₂O) δ 1.79-1.84 (m, 1H), 1.92-1.97 (m, 1H), 2.09-2.21 (m, 4H),2.14 (s, 3H), 3.12-3.19 (m, 1H), 6.59 (d, J=7.5 Hz, 1H), 6.68 (dd, J=7.3, 7.2 Hz, 1H), 7.01-7.04 (m, 2H).

### EXAMPLE 56

### N′-(2-methylphenyl)cyclopentanecarbohydrazide

The title compound was prepared using the procedure as described in Example 5, substituting cyclopentanecarboxylic acid for butyric acid. MS (ESI⁺) m/z 219.0 (M+H)⁺; ¹HNMR (DMSO-d₆/D₂O) δ 1.53-1.57 (m, 2H), 1.62-1.70 (m, 4H), 1.81-1.86 (2H), 2.14 (s, 3H), 2.66-2.72 (m, 1H), 6.62 (d, J=8.1 Hz, 1H), 6.68 (dd, J=7.5, 7.5 Hz, 1H), 7.01-7.04 (m, 2H).

### EXAMPLE 57

### N′-(2-methylphenyl)cyclohexanecarbohydrazide

The title compound was prepared using the procedure as described in Example 5, substituting cyclohexanecarboxylic acid for butyric acid. MS (ESI⁺) m/z 233.0 (M+H)⁺; ¹HNMR (DMSO-d₆/D₂O) δ 1.13-1.31 (m, 3H), 0.35-1.44 (m, 2H), 1.63-1.65 (m, 1H), 1.73-1.77 (m, 4H), 2.14 (s, 3H), 2.23-2.29 (m, 1H), 6.62 (d, J=6.9 Hz, 1H), 6.68 (dd, J=6.9, 6.9 Hz, 1H), 7.00-7.04 (m, 2H).

### EXAMPLE 58

### 2-methyl-N′-(2-methylphenyl)cyclohexanecarbohydrazide

The title compound was prepared using the procedure as described in Example 5, substituting 2-methylcyclohexanecarboxylic acid for butyric acid. MS (ESI⁺) m/z 247.0 (M+H)⁺; ¹HNMR (DMSO-d₆/D₂O) δ 0.90 (d, J=6.9 Hz, 3H), 1.26-1.35 (m, 2H), 1.45-1.50 (m, 3H), 1.61-1.72 (m, 3H), 2.02-2.05 (m, 1H), 2.15 (s, 3H), 2.44-2.48 (m, 1H), 6.65-6.69 (m, 2H), 7.00-7.04 (m, 2H).

### EXAMPLE 59

### 3-methyl-N′-(2-methylphenyl)cyclohexanecarbohydrazide

The title compound was prepared using the procedure as described in Example 5, substituting 3-methylcyclohexanecarboxylic acid for butyric acid. MS (ESI⁺) m/z 247.0 (M+H)⁺.

### EXAMPLE 60

### 4-methyl-N′-(2-methylphenyl)cyclohexanecarbohydrazide

The title compound was prepared using the procedure as described in Example 5, substituting 4-methylcyclohexanecarboxylic acid for butyric acid. MS (ESI⁺) m/z 247.1 (M+H)⁺.

### EXAMPLE 61

### N′-(2-methylphenyl)cycloheptanecarbohydrazide

The title compound was prepared using the procedure as described in Example 5, substituting cycloheptanecarboxylic acid for butyric acid. MS (ESI⁺) m/z 247.0 (M+H)⁺; ¹HNMR (DMSO-d₆/D₂O) δ 1.41-1.68 (m, 8H), 1.69-1.74 (m, 2H), 1.77-1.83 (m, 2H), 2.14 (S, 3H), 2.39-2.44 (m, 1H), 6.62 (d, J=7.8 Hz, 1H), 6.68 (dd, J=6.9, 6.9 Hz, 1H), 7.00-7.04 (m, 2H).

### EXAMPLE 62

### 2-bicyclo[2.2.1]hept-2-yl-N′-(2-methylphenyl)acetohydrazide

The title compound was prepared using the procedure as described in Example 5, substituting bicyclo[2.2.1]hept-2-ylacetic acid for butyric acid. MS (ESI⁺) m/z 259.0 (M+H)⁺; ¹HNMR (DMSO-d₆/D₂O) δ 1.09-1.17 (m, 4H), 1.35-1.52 (m, 4H), 1.82-1.87 (m, 1H), 2.00-2.05 (m, 2H), 2.14 (s, 3H), 2.13-2.17 (m, 1H), 2.20 (br s, 1H), 6.63 (d, J=7.8 Hz, 1H), 6.68 (dd, J=7.3, 7.2 Hz, 1H), 7.01-7.04 (m, 2H).

### EXAMPLE 63

### 1-methyl-N′-(2-methylphenyl)cyclopropanecarbohydrazide

The title compound was prepared using the procedure as described in Example 5, substituting 1-methylcyclopropanecarboxylic acid for butyric acid. MS (ESI⁺) m/z 205.1 (M+H)⁺; ¹HNMR (DMSO-d₆/D₂O) δ 0.60-0.62 (m, 2H), 1.00-1.02 (m, 2H), 1.36 (s, 3H), 2.15 (s, 3H), 6.64 (d, J=7.8 Hz, 1H), 6.68 (dd, J-7.3, 7.2 Hz, 1H), 7.00-7.04 (m, 2H).

### EXAMPLE 64

### 2-methyl-N′-(2-methylphenyl)cyclopropanecarbohydrazide

The title compound was prepared using the procedure as described in Example 5, substituting 2-methylcyclopropanecarboxylic acid for butyric acid. MS (ESI⁺) m/z 204.9 (M+H)⁺; ¹HNMR (DMSO-d₆/D₂O) δ 0.60-0.63 (m, 1H), 0.90-0.97 (m, 1H), 1.09 (d, J-5.9 Hz, 3H), 1.12-1.18 (m, 1H), 1.41-1.45 (m, 1H), 2.13 (s, 3H), 6.64 (d, J=8.1 Hz, 1H), 6.67 (dd, J=7.3, 7.2 Hz, 1H), 7.00-7.05 (m, 2H).

### EXAMPLE 65

### 2-(benzyloxy)-N′-(2-methylphenyl)acetohydrazide

The title compound was prepared using the procedure as described in Example 5, substituting benzyloxyacetic acid for butyric acid. MS (ESI⁺) m/z 270.9 (M+H)⁺; ¹HNMR (DMSO-d₆/D₂O) δ 2.16 (s, 3H), 4.07 (s, 2H), 4.61 (s, 2H), 6.64 (d, J=8.4 Hz, 1H), 6.70 (dd, J=7.3, 7.2 Hz, 1H), 7.03 (dd, J=6.7, 6.7 Hz, 1H), 7.32-7.36 (m, 3H), 7.38-7.43 (m, 3H).

### EXAMPLE 66

### 2-(3-methylphenoxy)-N′-(2-methylphenyl)acetohydrazide

The title compound was prepared using the procedure as described in Example 5, substituting m-tolyloxyacetic acid for butyric acid. MS (ESI⁺) m/z 271.0 (M+H)⁺; ¹HNMR (DMSO-d₆/D₂O) δ 2.30 (S, 3H), 2.16 (s, 3H), 4.63 (s, 2H), 6.61 (d, J=7.8 Hz, 1H), 6.69 (dd, J=7.3, 7.2 Hz, 1H), 6.80-6.84 (m, 3H), 6.99 (dd, J=7.6, 7.5 Hz, 1H), 7.02 (d, J=7.5 Hz, 1H), 7.21 (dd, J=7.8, 7.8 Hz, 1H).

### EXAMPLE 67

### 2-(2-methylphenoxy)-N′-(2-methylphenyl)acetohydrazide

The title compound was prepared using the procedure as described in Example 5, substituting o-tolyloxyacetic acid for butyric acid. MS (ESI⁺) m/z 270.9 (M+H)⁺; ¹HNMR (DMSO-d₆/D₂O) δ 2.16 (s, 3H), 2.25 (s, 3H), 4.67 (s, 2H), 6.63 (d, J=7.8 Hz, 1H), 6.70 (dd, J=6.7, 6.7 Hz, 1H), 6.90-6.92 (m, 2H), 6.98-7.03 (m, 2H), 7.17-7.19 (m, 2H).

### EXAMPLE 68

### 2-(4-methylphenoxy)-N′-(2-methylphenyl)acetohydrazide

The title compound was prepared using the procedure as described in Example 5, substituting p-tolyloxyacetic acid for butyric acid. MS (ESI⁺) m/z 270.9 (M+H)⁺; ¹HNMR (DMSO-d₆/D₂O) δ 2.16 (s, 3H), 2.26 (s, 3H), 4.62 (s, 2H), 6.61 (d, J=8.1 Hz, 1H), 6.70 (dd, J=7.3, 7.2 Hz, 1H), 6.93 (d, J=8.4 Hz, 2H), 7.00 (dd, J=7.6, 7.5 Hz, 1H), 7.03 (d, J=7.5 Hz, 1H), 7.14 (d, J=8.4 Hz, 2H).

### EXAMPLE 69

### 1-acetyl-N′-(2-methylphenyl)piperidine-4-carbohydrazide

The title compound was prepared using the procedure as described in Example 5, substituting 1-acetyl-piperidine-4-carboxylic acid for butyric acid. MS (ESI⁺) m/z 276.0 (M+H)⁺; ¹HNMR (DMSO-d₆/D₂O) δ 1.39-1.47 (m, 1H), 1.53-1.61 (m, 1H), 1.76-1.82 (m, 2H), 2.01 (s, 3H), 2.14 (s, 3H), 2.50-2.54 (m, 1H), 2.60-2.64 (m, 1H), 3.07-3.11 (m, 1H), 3.86 (d, J=13.4 Hz, 1H), 4.38 (d, J=13.1 Hz, 1H), 6.62 (d, J=8.1 Hz, 1H), 6.69 (dd, J=6.9, 6.9 Hz, 1H), 7.01-7.04 (m, 2H).

### EXAMPLE 70

### N′-(2,3-dichlorophenyl)adamantane-1-carbohydrazide

The title compound was prepared using the procedure as described in Example 1, substituting 2,3-dichlorophenylhydrazine hydrochloride for the hydrochloride salt of o-tolylhydrazine. MS (ESI⁺) m/z 340.9 (M+H)⁺; ¹HNMR (CDCl₃) δ 1.71-1.82 (m, 6H), 1.95-1.96 (m, 6H), 2.09 (br s, 3H), 6.47 (br s, 1H), 6.73 (dd, J=8.1, 1.7 Hz, 1H), 6.99 (dd, J=8.1, 1.7 Hz, 1H), 7.07 (t, J=8.0 Hz, 1H), 7.40 (br s, 1H). Anal. calc'd for C₁₇H₂₀Cl₂N₂O: C, 60.18; H, 5.94; N, 8.26. Found: C, 60.37; H, 5.91; N, 8.12.

### EXAMPLE 71

### N′-(2-chlorophenyl)adamantane-1-carbohydrazide

The title compound was prepared using the procedure as described in Example 1, substituting hydrochloride salt of 2-chlorophenylhydrazine for the hydrochloride salt of o-tolylhydrazine. MS (ESI⁺) m/z 305.0 (M+H)⁺; ¹HNMR (CDCl₃) δ 1.71-1.81 (m, 6H), 1.95-1.96 (m, 6H), 2.09 (br s, 1H), 6.80-6.86 (m, 1H), 7.11-7.17 (m, 1H), 7.26-7.29 (m, 1H), 7.39 (br s, 1H). Anal. calc'd for C₁₇H₂₁ClN₂O: C, 66.99; H, 6.94; N, 9.19. Found: C, 66.96; H, 7.13; N, 9.16.

### EXAMPLE 72

### N′-phenyladamantane-1-carbohydrazide

The title compound was prepared using the procedure as described in Example 1, substituting hydrochloride salt of phenylhydrazine for the hydrochloride salt of o-tolylhydrazine. MS (ESI⁺) m/z 271.1 (M+H)⁺; ¹HNMR (CDCl₃) δ 1.66-1.72 (m, 6H), 1.86-1.87 (m, 6H), 1.99 (br s, 3H), 6.69-6.73 (m, 3H), 7.12-7.15 (m, 2H).

### EXAMPLE 73

### N′-(pentafluorophenyl)adamantane-1-carbohydrazide

The title compound was prepared using the procedure as described in Example 1, substituting the hydrochloride salt of pentafluorophenylhydrazine for the hydrochloride salt of o-tolylhydrazine. MS (ESI⁺) m/z 361.0 (M+H)⁺; ¹HNMR (CDCl₃) δ 1.63-1.70 (m, 6H), 1.78-1.79 (m, 6H), 1.97 (br s, 3H).

### EXAMPLE 74

### N′-(2,5-dichlorophenyl)adamantane-1-carbohydrazide

The title compound was prepared using the procedure as described in Example 1, substituting the hydrochloride salt of 2,5-dichlorophenylhydrazine for the hydrochloride salt of o-tolylhydrazine. MS (ESI⁺) m/z 339.3 (M+H)⁺; ¹HNMR (CDCl₃) δ 1.67-1.73 (m, 6H), 1.87-1.88 (m, 6H), 2.01 (br s, 3H), 6.61 (d J=2.2 Hz, 1H), 6.80 (dd, J=8.6, 2.3 Hz, 1H), 7.32 (d, J=8.4 Hz, 1H).

### EXAMPLE 75

### N′-(2,4-dichlorophenyl)adamantane-1-carbohydrazide

The title compound was prepared using the procedure as described in Example 1, substituting the hydrochloride salt of 2,4-dichlorophenylhydrazine for the hydrochloride salt of o-tolylhydrazine. MS (ESI⁺) m/z 340.9 (M+H)⁺; ¹HNMR (CDCl₃) δ 1.66-1.73 (m, 6H), 1.86-1.87 (m, 6H), 2.00 (br s, 3H), 6.70 (d, J=8.7 Hz, 1H), 7.22 (dd, J=8.7,2.2 Hz, 1H), 7.40 (d, J=2.2 Hz, 1H).

### EXAMPLE 76

### N′-(3,4-dichlorophenyl)adamantane-1-carbohydrazide

The title compound was prepared using the procedure as described in Example 1, substituting the hydrochloride salt of 3,4-dichlorophenylhydrazine for the hydrochloride salt of o-tolylhydrazine. MS (ESI⁺) m/z 338.9 (M+H)⁺; ¹HNMR (CDCl₃) δ 1.66-1.72 (m, 6H), 1.85-1.86 (m, 6H), 2.00 (br s, 3H), 6.67 (dd, J=8.7,2.5 Hz, 1H), 6.81 (d, J=2.8 Hz, 1H), 7.35 (d,J=8.7 Hz, 1H).

### EXAMPLE 77

### N′-(4-fluorophenyl)adamantane-1-carbohydrazide

The title compound was prepared using the procedure as described in Example 1, substituting the hydrochloride salt of 4-fluorophenylhydrazine for the hydrochloride salt of o-tolylhydrazine. MS (ESI⁺) m/z 288.9 (M+H)⁺; ¹HNMR (CDCl₃) δ 1.66-1.72 (m, 6H), 1.85-1.86 (m, 6H), 1.99 (br s, 3H), 6.69-6.71 (m, 2H), 6.97 (dd, J=8.9, 8.8 Hz, 2H).

### EXAMPLE 78

### N′-(4-methoxyphenyl)adamantane-1-carbohydrazide

The title compound was prepared using the procedure as described in Example 1, substituting the hydrochloride salt of 4-methoxyphenylhydrazine for the hydrochloride salt of o-tolylhydrazine. MS (ESI⁺) m/z 301.0 (M+H)⁺; ¹HNMR (CDCl₃) δ 1.66-1.72 (m, 6H), 1.84-1.85 (m, 6H), 1.99 (br s, 3H), 3.66 (s, 3H), 6.68 (d, J=9.1 Hz, 2H), 6.76 (d, J=9.0 Hz, 2H).

### EXAMPLE 79

### N′-(2,5-dimethylphenyl)adamantane-1-carbohydrazide

The title compound was prepared using the procedure as described in Example 1, substituting the hydrochloride salt of 2,5-dimethylphenylhydrazine for the hydrochloride salt of o-tolylhydrazine. MS (ESI⁺) m/z 299.0 (M+H)⁺; ¹HNMR (CDCl₃) δ 1.67-1.73 (m, 6H), 1.88-1.89 (m, 6H), 2.01 (br s, 3H), 2.10 (s, 3H), 2.17 (s, 3H), 6.43 (s, 1H), 6.50 (d, J=7.5 Hz, 1H), 6.88 (d, J=7.5 Hz, 1H).

### EXAMPLE 80

### N′-(4-cyanophenyl)adamantane-1-carbohydrazide

The title compound was prepared using the procedure as described in Example 1, substituting the hydrochloride salt of 4-cyanophenylhydrazine for the hydrochloride salt of o-tolylhydrazine. MS (ESI⁺) m/z 296.0 (M+H)⁺; ¹HNMR (CDCl₃) δ 1.67-1.73 (m, 6H), 1.87-1.88 (m, 6H), 2.00 (br s, 3H), 6.73 (d, J=8.7 Hz, 2H), 7.54 (d, J=8.7 Hz, 2H).

### EXAMPLE 81

### N′-(2-fluorophenyl)adamantane-1-carbohydrazide

The title compound was prepared using the procedure as described in Example 1, substituting the hydrochloride salt of 2-fluorophenylhydrazine for the hydrochloride salt of o-tolylhydrazine. MS (ESI⁺) m/z 289.0 (M+H)⁺; ¹HNMR (CDCl₃) δ 1.66-1.72 (m, 6H), 1.86-1.87 (m, 6H), 2.00 (br s, 3H), 6.71-6.77 (m, 2H), 7.00 (dd, J=7.8, 7.8 Hz, 1H), 7.04-7.08 (m, 1H).

### EXAMPLE 82

### N′-(3-fluorophenyl)adamantane-1-carbohydrazide

The title compound was prepared using the procedure as described in Example 1, substituting the hydrochloride salt of 3-fluorophenylhydrazine for the hydrochloride salt of o-tolylhydrazine. MS (ESI⁺) m/z 289.1 (M+H)⁺; ¹HNMR (CDCl₃) δ 1.66-1.721.86-1.87 (m, 6H), 2.00 (br s, 3H), 6.38-6.41 (m, 1H), 6.46-6.50 (m, 1H), 6.53 (dd, J=8.0, 1.7 Hz, 1H), 7.13-7.17 (m, 1H).

### EXAMPLE 83

### N′-(4-methylphenyl)adamantane-1-carbohydrazide

The title compound was prepared using the procedure as described in Example 1, substituting the hydrochloride salt of 4-methylphenylhydrazine for the hydrochloride salt of o-tolylhydrazine. MS (ESI⁺) m/z 285.0 (M+H)⁺; ¹HNMR (CDCl₃) δ 1.68-1.72 (m, 6H), 1.84-1.85 (m, 6H), 1.99 (br s, 3H), 2.18 (s, 3H), 6.62 (d, J=8.4 Hz, 2H), 6.95 (d, J=8.1 Hz, 2H).

### EXAMPLE 84

### N′-[3-(trifluoromethyl)phenyl]adamantane-1-carbohydrazide

The title compound was prepared using the procedure as described in Example 1, substituting the hydrochloride salt of 3-trifluoromethylphenylhydrazine for the hydrochloride salt of o-tolylhydrazine. MS (ESI⁺) m/z 339.2 (M+H)⁺; ¹HNMR (CDCl₃) δ 1.67-1.73 (m, 6H), 1.86-1.87 (m, 6H), 2.00 (br s, 3H), 6.94-6.95 (m, 2H), 7.02 (d, J=7.8 Hz, 1H), 7.37 (dd, J=7.8, 7.8 Hz, 1H).

### EXAMPLE 85

### N′-quinolin-5-yladamantane-1-carbohydrazide

### EXAMPLE 85A

### 5-hydrazinoquinoline

To an oven-dried, round-bottomed flask containing a magnetic stir bar was added solid 5-aminoquinoline (5.05 g, 35.0 mmol). The flask was cooled to 0° C. in an ice bath and concentrated hydrochloric acid (30 mL) was added dropwise. A solution of sodium nitrite (2.42 g, 38.5 mmol) in water (10 mL) was added dropwise to the cold reaction slurry. The reaction mixture was stirred at 0° C. for 30 minutes and allowed to warm to room temperature over 30 minutes during which an orange/red solution formed. The flask was again cooled to 0° C. and a solution of tin(II) chloride dihydrate (15.8 g, 70.0 mmol) dissolved in the minimum amount of concentrated hydrochloric acid was added dropwise. A yellow precipitate formed immediately upon addition of the tin salt. The mixture was stirred at 0° C. for 30 minutes and then allowed to warm to room temperature with vigorous stirring over 4 hours. The solid yellow product was collected by vacuum filtration on a glass frit. The product was washed with cold ethanol and dried under vacuum to give 6.18 g of the title compound as a bis hydrogen chloride salt (76%). MS (DCI/NH₃) m/z 375 (M+H)⁺.

### EXAMPLE 85B

### N′-quinolin-5-yladamantane-1-carbohydrazide

To an oven-dried, 250-mL, round-bottomed flask containing a magnetic stir bar was added the product of Example 85A (1.16 g, 5.00 mmol). The flask was sealed with a septum and purged with dry nitrogen atmosphere. Anhydrous tetrahydrofuran (50 mL) was added via syring to form a golden colored slurry. Triethylamine (5.58 mL, 40.0 mmol) was added via syringe. The reaction flask was cooled to 0° C. and a solution of 1-chlorocarbonyladamantane (0.993 g, 5.00 mmol) in anhydrous tetrahydrofuran (5 mL) was added to the reaction mixture via syringe. The mixture was stirred at 0° C. for 30 minutes and then allowed to warm to room temperature for 2 hours. The reaction was monitored by LC-MS till completion. Quenched with water (50 mL) and extracted with dichloromethane (3×30 mL). The combined organic extracts were dried over magnesium sulfate, filtered, and concentrated to give an orange/brown solid. The product was purified by re-crystallization from ethyl acetate/hexanes to give 1.18 g (73%) of the title product. MS (ESI) m/z 321.9 (M+H)⁺; NMR (DMSO-d₆) δ 1.69-1.74 (m, 6H), 1.97-2.02 (m, 6H), 2.02-2.05 (m, 3H), 6.66 (d, J=6.8 Hz, 1H), 7.36 (d, J=8.1 Hz, 1H), 7.43 (dd, J=8.6, 4.2 Hz, 1H), 7.55-7.50 (m, 1H), (dd, J=8.34 (d, J=2.4 H, 1H), 8.68 (d, J=8.8 Hz, 1H), 8.83 (dd, J=4.1, 1.7 Hz, 1H), 9.61 (d, J=2.3 Hz, 1H). Anal. calcd for C₂₀H₂₃N₃O: C, 74.74; H, 7.21; N, 13.07. Found: C, 74.00; H, 7.09; N, 12.92.

### EXAMPLE 86

### N′-isoquinolin-5-yladamantane-1-carbohydrazide

### EXAMPLE 86A

### 5-hydrazinoisoquinoline

The bis hydrogen chloride salt of the title compound was prepared using the procedure as described in Example 85A, substituting 5-aminoisoquinoline for 5-aminoquinoline.

### EXAMPLE 86B

### N!-isoquinolin-5-yladamantane-1-carbohydrazide

The product of Example 86A (232 mg, 1.00 mmol) was reacted with 1-chlorocarbonyladamantane (199 mg, 1.00 mmol) according to the procedure as described in Example 1B to provide 99 mg (31%) of the title compound as a yellow solid. MS (ESI) m/z 322.0 (M+H)⁺; NMR (DMSO-d₆) δ 1.72 (br s, 6H), 1.94-1.95 (m, 6H), 2.02-203 (m, 3H), 6.80 (d, J=7.1, 1.4 Hz, 1H), 7.41-7.49 (m, 2H), 8.09 (d, J=6.1 Hz, 1H), 8.34 (d, J=2.4 Hz, 1H), 8.43 (d, J=6.1 Hz, 1H), 9.18 (s, 1H), 9.62 (d, J=2.4 Hz, 1H).

### EXAMPLE 87

### N′-(2-chloroquinolin-5-yl)adamantane-1-carbohydrazide

### EXAMPLE 87A

### 2-chloro-5-hydrazinoquinoline

The bis hydrogen chloride salt of the title compound was prepared using the procedure as described in Example 85A, substituting 2-chloro5-aminoquinoline (prepared according to the procedure as described in: Capps, J. D.; Hamoltion, C. S. J. Am. Chem. Soc. Vol. 60 pp. 2104 (1938)) for 5-aminoquinoline.

### EXAMPLE 87B

### N′-(2-chloroquinolin-5-yl)adamantane-1-carbohydrazide

The product of Example 87A (533 mg, 2.00 mmol) was reacted with 1-chlorocarbonyladamantane (397 mg, 2.00 mmol) according to the procedure as described in Example 85B to provide 109 mg (15%) of the title compound as a yellow solid. MS (ESI) m/z 356.1 (M+H)⁺; NMR (DMSO-d₆) δ 1.72 (br s, 6H), 1.93-1.94 (m, 6H), 2.02 (br s, 3H), 6.69 (d, J=7.5 Hz, 1H), 7.28 (d, J=8.1 Hz, 1H), 7.51 (d, J=9.2 Hz, 1H), 7.59 (dd, J=8.1, 8.0 Hz, 1H), 8.52 (d, J=2.0 Hz, 1H), 8.73 (d, J=8.8 Hz, 1H), 9.64 (d, J=2.0 Hz, 1H).

### EXAMPLE 88

### 2-(1-adamantyl)-N′-quinolin-5-ylacetohydrazide

The title compound was prepared using the procedure of Example 85B, reacting the product of Example 85A (464 mg, 2.00 mmol) with adamantan-1-yl-acetyl chloride (425 mg, 2.00 mmol). MS (ESI) m/z 336.0 (M+H)⁺; NMR (DMSO-d₆) δ 1.59-1.71 (m, 1H), 1.96 (br s, 5H), 2.01 (s, 2H), 7.37 (d, J=8.1 Hz, 1H), 7.44 (dd, J=8.6, 4.2 Hz, 1H), 7.53 (dd, J=8.1, 8.0 Hz, 1H), 8.41 (d, J=2.4 Hz, 1H), 8.66 (d, J=8.8 Hz, 1H), 8.83 (dd, J=4.1, 1.7 Hz, 1H), 9.70 (d, J=2.0 Hz, 1H).

### EXAMPLE 89

### 3-(1-adamantyl)-N′-quinolin-5-ylpropanohydrazide

The product of Example 85A (464 mg, 2.00 mmol) was reacted with 3-(1-adamantyl)propanoyl chloride (425 mg, 2.00 mmol) according to the procedure as described in Example 85B to provide 156 mg (22%) of the title compound as a white solid. MS (ESI) m/z 350.0 (M+H)⁺; NMR (DMSO-d₆) δ 1.36-1.42 n(m, 2H), 1.48-1.49 (m, 6H), 1.60-1.72 (m, 6H), 1.85 (br s, 3H), 2.18-2.24 (m, 2H), 6.71 (dd, J=7.6, 0.9 Hz, 1H), 7.37 (d, J=8.5 Hz, 1H), 7.44 (dd, J=8.6, 4.2 Hz, 1H), 7.52 (dd, J=8.1, 8.0 Hz, 1H), 8.41 (d, J=2.0 Hz, 1H), 8.63 (d, J=8.1 Hz, 1H), 8.83 (dd, J=4.2, 1.5 Hz, 1H), 9.80 (d, J=2.0 Hz, 1H).

### EXAMPLE 90

### N′-quinolin-5-ylhexahydro-2,5-methanopentalene-3a(1 H)-carbohydrazide

The product of Example 85A (812 mg, 3.50 mmol) was reacted with noradamantan-3-carbonyl chloride (739 mg, 4.00 mmol) according to the procedure as described in Example 85B to provide 692 mg (64%) of the title compound as a yellow solid. MS (ESI) m/z 308.0 (M+H)⁺; NMR (DMSO-d₆) δ 1.59-1.84 (m, 4H), 1.78-1.84 (m, 2H), 1.89-1.93 (m, 2H), 2.03-2.07 (m, 2H), 2.30 (br s, 2H), 2.69-2.74 (m, 1H), 6.70 (d, J=7.5 Hz, 1H), 7.37 (d, J=8.5 Hz, 1H), 7.44 (8.6, 4.2 Hz, 1H), 7.54 (dd, =8.4, 8.1 Hz, 1H), 8.41 (d, J=2.0 Hz, 1H), 8.67 (d, J=6.8 Hz, 1H), 8.84 (dd, J=4.1 1.7 Hz, 1H), 9.61 (d, J=2.0 Hz, 1H).

### EXAMPLE 91

### 3-chloro-N′-quinolin-5-yladamantane-1-carbohydrazide

The product of Example 85A (1.39 g, 6.00 mmol) was reacted with 3-chloroadamantane-1-carbonyl chloride (1.17 g, 5.00 mmol) according to the procedure as described in Example 85B to provide 1.38 g (78%) of the title compound as a yellow solid. MS (ESI) m/z 355.9 (M+H)⁺; NMR (DMSO-d₆) δ 1.63-1.69 (m, 2H), 1.92 (br s, 4H), 2.11-2.12 (m, 4H), 2.26 (br s, 2H), 2.30 (br s, 2H), 6.66 (d, J=7.5 Hz, 1H), 7.38 (d, J=8.1 Hz, 1H), 7.44 (dd, J=8.6, 4.2 Hz, 1H), 7.53 (t, J=8.0 Hz, 1H), 8.39 (s, 1H), 8.67 (d, 8.8 Hz, 1H), 8.84 (dd, J=4.1, 1.4 Hz, 1H), 9.75 (d, J=2.0 Hz, 1H).

### EXAMPLE 92

### 3-bromo-N′-quinolin-5-yladamantane-1-carbohydrazide

The product of Example 85A (464 mg, 2.00 mmol) was reacted with 3-bromoadamantane-1-carbonyl chloride (555 mg, 2.00 mmol) according to the procedure as described in Example 85B to provide 139 mg (17%) of the title compound as a yellow solid. MS (ESI) m/z 402.2 (M+H)⁺; NMR (DMSO-d₆) δ 1.70-1.72 (m, 2H), 1.98 (br s, 4H), 2.22 (br s, 2H), 2.27-2.28 (m, 1H), 2.32-2.33 (m, 4H), 2.52 (br s, 1H), 6.66 (d, J=6.8 Hz, 1H), 7.37 (d, J=8.5 Hz, 1H), 7.44 (d, J=8.5, 4.1 Hz, 1H), 7.51-7.78 (m, 1H), 8.39 (d, J=2.0, 1H), 8.66 (d, J=8.8 Hz, 1H), 8.84 (dd, J=4.1, 1.7 Hz, 1H), 9.74 (d, J=2.4 Hz, 1H).

### EXAMPLE 93

### 3-ethyl-N′-quinolin-5-yladamantane-1-carbohydrazide

To an oven-dried flask containing a magnetic stir bar were added the product of Example 85A (139 mg, 0.600 mmol), 3-ethyladamantane-1-carboxylic acid (104 mg, 0.500 mmol) and 2-(1-H-benzotriazol-1-yl-1,1,3,3-tetramethyluronium tetrafluoroborate (193 mg, 0.600 mmol). The flask was sealed with a septum and anhydrous acetonitrile (4 mL) and dimethylformamide (1 mL) were added via syringe to form a white colored slurry. Triethylamine (488 μL, 3.50 mmol) was added via syringe and the reaction was stirred at room temperature for 12 h. Quenched with water (10 mL) and extracted with dichloromethane (3×8 mL). The combined organic extracts were dried over magnesium sulfate, filtered, and concentrated to give a brown oil. The product was purified by preparative HPLC on a Waters Symmetry C8 column (40mm×100 mm, 7 μm particle size) using a gradient of 10% to 100% acetonitrile: 10 mM ammonium acetate over 12 minutes (15 minute run time) at a flow rate of 70 mL/minute. MS (ESI) m/z 350.1 (M+H)⁺; NMR (DMSO-d₆) δ 0.81 (t, J=7.5 Hz, 3H), 1.16 (q, J=7.2 Hz, 2H), 1.44 (br s, 4H), 1.58-1.69 (m, 4H), 1.81-1.93 (m, 4H), 2.09 (br s, 2H), 6.66 (d, J=7.5 HZ, 1H), 7.36 (d, J=8.5 Hz, 1H), 7.44 (dd, J=8.5, 4.1 Hz, 1H), 7.53 (dd, J=8.1, 8.0 Hz, 1H), 8.34 (d, J=2.0 Hz, 1H), 8.68 (d, J=8.1 Hz, 1H), 8.83 (dd, J=4.1, 1.7 Hz, 1H), 9.61 (d, J=2.4 Hz, 1H).

### EXAMPLE 94

### 3,5-dimethyl-N′-quinolin-5-yladamantane-1-carbohydrazide

The product of Example 85A (139 mg, 0.600 mmol) was reacted with 3,5-dimethyladamantane-1-carboxylic acid (104 mg, 0.500 mmol) according to the procedure as described in Example 93 to provide 21.3 mg of the title compound as a white solid. MS (ESI) m/z 350.1 (M+H)⁺; NMR (DMSO-d₆) δ 0.86 (s, 6H), 1.17 (br s, 2H), 1.31-1.42 (m, 4H), 1.51-1.62 (m, 4H), 1.78 (d, J=2.7 Hz, 2H), 2.08-2.13 (m, 1H), 6.64 (d, J=6.8 Hz, 1H), 7.36 (d, J=8.1 Hz, 1H), 7.43 (dd, J=8.5, 4.1 Hz, 1H), 7.53 (dd, J=8.1, 8.0 Hz, 1H), 8.33 (d, J=2.4 Hz, 1H), 8.67 (d, J=7.8 Hz, 1H), 8.83 (dd, J=4.2, 1.5 Hz, 1H), 9.59 (d, J=2.4 Hz, 1H).

### EXAMPLE 95

### 3-(1,1,2,3,3,3-hexafluoropropyl)-N′-quinolin-5-yladamantane-1-carbohydrazide

The product of Example 85A (139 mg, 0.600 mmol) was reacted with 3-(1,1,2,3,3,3-hexafluoro-propyl)-adamantane-1-carboxylic acid (165 mg, 0.500 mmol) according to the procedure as described in Example 93 to provide 29.0 mg of the title compound as a white solid. MS (ESI) m/z 472.1 (M+H)⁺; NMR (DMSO-d₆) δ 1.70-1.76 (m, 6H), 1.86-2.03 (m, 6H), 2.21 (br s, 2H), 6.00-6.26 (m, 1H), 6.67 (d, J=6.8 Hz, 1H), 7.37 (d, J=8.1 Hz, 1H), 7.44 (dd, J=8.6, 4.2 Hz, 1H), 7.50-7.55 (m, 1H), 8.38 (d, J=2.0 Hz, 1H), 8.67 (d, J=8.1 Hz, 1H), 8.84 (dd, J=4.2, 1.5 Hz,), 9.73 (D, J=2.0 Hz, 1H).

### EXAMPLE 96

### 1-methyl-2,2-diphenyl-N′-quinolin-5-ylcyclopropanecarbohydrazide

The product of Example 85A (139 mg, 0.600 mmol) was reacted with 1-methyl-2,2-diphenyl-cyclopropanecarboxylic acid (126 mg, 0.500 mmol) according to the procedure as described in Example 93 to provide 18.2 mg of the title compound as a white solid. MS (ESI) m/z 394.1 (M+H)⁺; NMR (DMSO-d₆) δ 1.28 (S, 3H).1.35 b(d, J=5.4 Hz, 1H), 2.36 (d, J=5.1 Hz, 1H), (d, J=7.5 Hz, 1H), 7.14-7.35 (m, 8H), 7.39 (dd, J=8.6, 4.2 Hz, 1H), 7.51-7.59 (m, 4H), 8.21 (d, J=1.7 Hz, 1H), 8.60 (d, J=8.5 Hz, 1H), 8.79 (dd J=4.1, 1.4 Hz, 1H), H =9.85 (d, J=1.7 Hz, 1H).

### EXAMPLE 97

### 2,2,3,3-tetramethyl-N′-quinolin-5-ylcyclopropanecarbohydrazide

The product of Example 85A (139 mg, 0.600 mmol) was reacted with 1-methyl-2,2-diphenyl-cyclopropanecarboxylic acid (71.1 mg, 0.500 mmol) according to the procedure of Example 93 to provide 18.2 mg of the title compound as a white solid. MS (ESI) m/z 394.1 (M+H)⁺; NMR (CDCl₃) δ 1.26 (s, 6H), 1.31 (s, 6H), 2.5 (br s, 1H), 7.00 (d, J=7.5 Hz, 1H), 7.12 (br s, 1H), 7.28 (dd, J=8.4, 6.1 Hz, 1H), 7.44 (br s, 1H), 7.56 (dd, J=8.1, 8.1 Hz, 1H), 7.66 (d, J=8.4 Hz, 1H), 8.32 (d, J=8.5 Hz, 1H), 8.84 (d, J=3.7 Hz, 1H).

### EXAMPLE 98

### 1-phenyl-N′-quinolin-5-ylcyclopropanecarbohydrazide

The product of Example 85A (464 mg, 2.00 mmol) was reacted with 1-phenyl-cyclopropanecarbonyl chloride (361 mg, 2.00 mmol) according to the procedure as described in Example 85B to provide 130 mg (21%) of the title compound as a yellow solid. MS (ESI) m/z 303.9 (M+H)⁺; NMR (DMSO-d₆) δ 1.08-1.12 (m, 2H), 1.42-1.45 (m, 2H), 6.60 (d, J=6.8 Hz, 1H), 7.28-7.54 (m, 8H), 8.39 (d, J=2.0 Hz, 1H), 8.60 (d, J=8.8 Hz, 1H), 8.81 (dd, J=4.2, 1.5 Hz, 1H), 9.1 (d, J=2.0 Hz, 1H).

### EXAMPLE 99

### N′-quinolin-5-yl-1-thien-2-ylcyclopropanecarbohydrazide

The product of Example 85A (139 mg, 0.600 mmol) was reacted with 1-thiophen-2-yl-cyclopropanecarboxylic acid (84.1 mg, 0.500 mmol) according to the procedure as described in Example 93 to provide 38.5 mg of the title compound as a white solid. MS (ESI) m/z 309.8 (M+H)⁺; NMR (DMSO-d₆) δ 1.18-1.24 (m, 2H), 1.51-1.57 (m, 2H), 6.66 (d, J=7.8 Hz, 1H), 7.02 (d, J=5.1, 3.7 Hz, 1H), 7.16 (d, J=3.4 Hz, 1H), 7.37 (d, J=8.5 Hz, 1H), 7.43 (dd, J=8.6, 4.2 Hz, 1H), 7.48-7.51 (m, 1H), 7.54 (d, J=8.5 Hz, 1H), 8.42 (d, J=1.4 Hz, 1H), 8.62 (d, J=8.5 Hz, 1H), 8.82 (dd, J=4.1, 1.4 Hz, 1H), 9.37 (d, J=1.4 Hz, 1H).

### EXAMPLE 100

### 1-cyclohexyl-N′-quinolin-5-ylcyclopropanecarbohydrazide

The product of Example 85A (139 mg, 0.600 mmol) was reacted with 1-cyclohexyl-cyclopropanecarboxylic acid (84.1 mg, 0.500 mmol) according to the procedure as described in Example 93 to provide 41.6 mg of the title compound as a white solid. MS (ESI) m/z 310.2 (M+H)⁺; NMR (CDCl₃) δ 0.78-0.82 (m, 2H), 1.04-1.08 (m, 2H), 1.09-1.35 (m, 5H), 1.50-1.59 (m, 1H) 1.69-1.74 (m, 1H), 1.80-1.85 (m, 4H), 6.90 (d, J=7.8 Hz, 1H), 7.08 (br s, 1H), 7.22-7.27 (m, 1H), 7.54 (dd, J=8.5, 8.1 Hz, 1H), 7.58 (br s, 1H), 7.65 (d, J=8.5 Hz, 1H), 8.25 (d, J=7.8 Hz, 1H), 8.83 (dd, J=4.4 1.7 Hz, 1H).

### EXAMPLE 101

### N′-quinolin-5-ylspiro[2.5]octane-1-carbohydrazide

The product of Example 85A (139 mg, 0.600 mmol) was reacted with 1-cyclohexyl-cyclopropanecarboxylic acid (77.1 mg, 0.500 mmol) according to the procedure as described in Example 93 to provide 17.9 mg of the title compound as a white solid. MS (ESI) m/z 296.1 (M+H)⁺; NMR (CDCl₃) δ 0.88-0.92 (m, 1H), 1.22-1.25 (m, 1H), 1.37-1.64 (m, 11 H), 7.00 (d, J=6.8 Hz, 1H),7.26 (brs, 1H), 7.23-7.27 (m, 1H), 7.53 (dd, J=8.1, 8.0 Hz, 1H), 7.63 (d, J=8.4 Hz, 1H), 7.73 (br s, 1H), 8.33 (d, J=8.5 Hz, 1H), 8.82(d, J=3.0 Hz, 1H).

### EXAMPLE 102

### 1-benzyl-N′-quinolin-5-ylcyclopentanecarbohydrazide

The product of Example 85A (139 mg, 0.600 mmol) was reacted with 1-benzyl-cyclopentanecarboxylic acid (102 mg, 0.500 mmol) according to the procedure as described in Example 93 to provide 29.5 mg of the title compound as a white solid. MS (ESI) m/z 345.9 (M+H)⁺; NMR (CDCl₃) δ 1.58-1.67 (m, 6H), 2.08-2.11 (m, 2H), 3.03 (s, 2H), 6.47 (d, J=6.4 Hz, 1H), 7.19-7.47 (m, 8H), 8.33 (d, J=2.0 Hz, 1H), 8.72 (d, J=7.8 Hz, 1H), 8.84 (dd, J=4.2, 1.5 Hz, 1H), 9.76 (d, J=2.0 Hz, 1H).

### EXAMPLE 103

### 1-(2-fluorophenyl)-N′-quinolin-5-ylcyclohexanecarbohydrazide

The product of Example 85A (464 mg, 2.00 mmol) was reacted with 1-(2-fluoro-phenyl)-cyclohexanecarbonyl chloride (445 mg, 2.00 mmol) according to the procedure as described in Example 85B to provide 312 mg (43%) of the title compound as a yellow solid. MS (ESI) m/z 363.9 (M+H)⁺; NMR (CDCl₃) δ 1.36-1.40 (m, 1H), 1.54-1.57 (m, 3H), 1.65-1.75 (m, 2H), 1.95-2.02 (m, 2H), 2.37-2.42 (m, 2H), 6.51 (d, J=6.8 Hz, 1H), 7.17-7.28 (m, 2H), 7.33-7.38 (m, 2H), 7.40-7.46 (m, 2H), 7.52-7.58 (m, 1H), 8.33 (br s, 1H), 8.70 (d, J=8.5 Hz, 1H), 8.83 (dd, J=4.1, 1.7 Hz, 1H), 9.48 (br s, 1H).

### EXAMPLE 104

### 1-(3-fluorophenyl)-N′-quinolin-5-ylcyclohexanecarbohydrazide

The product of Example 85A (464 mg, 2.00 mmol) was reacted with 1-(3-fluoro-phenyl)-cyclohexanecarbonyl chloride (445 mg, 2.00 mmol) according to the procedure as described in Example 85B to provide 291 mg (40%) of the title compound as a yellow solid. MS (ESI) m/z 363.9 (M+H)⁺; NMR (CDCl₃) δ 1.29-1.36 (m, 1H), 1.55-1.675 (m, 5H), 1.78-1.86 (m, 2H), 2.50-2.54 (m, 2H), 6.29 (d, J=6.9 Hz, 1H), 7.12-7.18 (m, 1H), 7.24-7.50 (m, 5H), 7.57 (dd, J=8.6, 4.2 H, 1H), H =8.58 (Br s, 1H), 8.85 (D, J=8.8 Hz, 1H), 8.93 (D, J=4.1 Hz, 1H), 9.78 (br s, 1H).

### EXAMPLE 105

### 1-(4-fluorophenyl)-N′-quinolin-5-ylcyclohexanecarbohydrazide

The product of Example 85A (464 mg, 2.00 mmol) was reacted with 1-(4-fluoro-phenyl)-cyclohexanecarbonyl chloride (445 mg, 2.00 mmol) according to the procedure of Example 85B to provide 173 mg (24%) of the title compound as a yellow solid. MS (ESI) m/z 363.9 (M+H)⁺; NMR (CDCl₃) δ 1.28-1.35 (m, 1H), 1.56-1.63 (m, 5H), 1.77-1.85 (m, 2H), 2.50-2.54 (m, 2H), 6.22-6.24 (m, 1H), 7.23-7.26 (m, 2H), 7.32-7.34 (m, 2H), 7.42 (dd, J=8.5, 4.1 Hz, 1H), 7.49-7.53 (m, 2H), 8.36 (s, 1H), 8.66 (d, J=8.5 Hz, 1H), 8.82 (dd, J=4.1, 1.4 Hz, 1H), 9.70 (br s, 1H).

### EXAMPLE 106

### 1-(4-methoxyphenyl)-N′-quinolin-5-ylcyclohexanecarbohydrazide

The product of Example 85A (464 mg, 2.00 mmol) was reacted with 1-(4-methoxy-phenyl)-cyclohexanecarbonyl chloride (758 mg, 3.00 mmol) according to the procedure as described in Example 85B to provide 302 mg (28%) of the title compound as a yellow solid. MS (ESI) m/z 376.2 (M+H)⁺; NMR (CDCl₃) δ 1.25-1.35 (m, 1H), 1.58-1.59 (m, 5H), 1.75-1.84 (m, 2H), 2.48-2.50 (m, 2H), 3.78 (s, 3H), 6.21-6.27 (m, 1H), 6.96 (d, J=8.8 Hz, 2H), 7.31 (d, J=m4.4 Hz, 2H), 7.37-7.43 (m, 3H), 8.32 (d, J=2.0 Hz, 1H), 8.65 (dd, J=8.5, 1.4 Hz, 1H), 8.81 (dd, J=4.2, 1.5 Hz, 1H), 9.60 (d, J=1.7 Hz, 1H).

### EXAMPLE 107

### 3-isopropyl-1-methyl-N′-quinolin-5-ylcyclopentanecarbohydrazide

The product of Example 85A (139 mg, 0.600 mmol) was reacted with 1-cyclohexyl-cyclopropanecarboxylic acid (105 mg, 0.500 mmol) according to the procedure as described in Example 93 to provide 23.9 mg (15%) of the title compound as a white solid. MS (ESI) m/z 312.2 (M+H)⁺; NMR (DMSO-d₆) δ 0.88 (d, J=8.6 Hz, 3H), 0.89 (d, J=8.6 Jz, 3H), 1.33 (s, 3H), 1.24-1.54 (m, 4H), 1.73-1.74 (m, 2H), 1.75-1.85 (m, 1H), 2.12-2.21 (m, 1H), 6.68 (d, J=7.1 HZ, 1H), 7.37 (d, J=8.5 HZ, 1H), 7.44 (dd, J=8.6, 4.2 Hz, 1H), 7.53 (dd, J=8.1, 8.0 Hz, 1H), 8.36 (d, =2.0 Hz, 1H), 8.67 (d, J=8.1 Hz, 1H), 8.83 (dd, J=4.2, 1.5 Hz, 1H), 9.66 (d, J=2.4 Hz, 1H).

### EXAMPLE 108

### (1R,3S)-1,2,2,3-tetramethyl-N′-quinolin-5-ylcyclopentanecarbohydrazide

The product of Example 85A (464 mg, 2.00 mmol) was reacted with D-campholic acid (340 mg, 2.00 mmol) according to the procedure as described in Example 93 to provide 135 mg (15%) of the title compound as a white solid. MS (ESI) m/z 311.9 (M+H)⁺; NMR (DMSO-d₆) δ 0.72 (s, 3H), 0.84 (d, J=6.4 Hz, 3H), 1.05 (s, 3H), 1.24 (s, 3H), 1.22-1.29 (m, 1H), 1.43-1.51 (m, 1H), 1.83-1.95 (m, 2H), 2.39-2.50 (m, 1H), 6.74 (d, J=7.5 Hz, 1H), 7.37 (d, J=8.1 Hz, 1H), 7.45 (dd, J=8.6, 4.2 Hz, 1H), 7.53 (dd, J=8.1, 8.0 HZ, 1H), 8.33 (D, J=2.4 Hz, 1H), 8.72 (d, J=8.5 Hz, 1H), 8.84 (dd, J=m 4.2, 1.5 Hz, 1H), 9.47 (d, J=2.4 Hz, 1H).

### EXAMPLE 109

### 1-methyl-N′-quinolin-5-ylindane-2-carbohydrazide

The product of Example 85A (139 mg, 0.600 mmol) was reacted with 1-cyclohexyl-cyclopropanecarboxylic acid (88.0 mg, 0.500 mmol) according to the procedure as described in Example 93 to provide 22.5 mg (14%) of the title compound as a white solid. MS (ESI) m/z 318.0 (M+H)⁺.

### EXAMPLE 110

### N′-quinolin-5-yldodecahydro-1H-fluorene-9-carbohydrazide

The product of Example 85A (139 mg, 0.600 mmol) was reacted with dodecahydro-fluorene-9-carboxylic acid (111 mg, 0.500 mmol) according to the procedure as described in Example 93 to provide 19.0 mg (10%) of the title compound as a white solid. MS (ESI) m/z 364.2 (M+H)⁺; NMR (CDCl₃) δ 1.22-1.34 (m, 5H), 1.49-1.79 (m, 12H), 2.07-2.18 (m, 2H), 2.32-2.43 (m, 2H), 2.95 (t, J=6.2 Hz, 1H), 6.97 (d, J=7.8 Hz, 1H), 7.22 (dd, J=8.5, 4.4 Hz, 1H), 7.50 (dd, J=8.1, 8.0 Hz, 1H), 7.59 (d, J=8.6 Hz, 1H), 7.66 (br s, 1H), 8.33 (D, J=8.5 Hz, 1H), 8.79 (d, J=4.1 Hz, 1H).

### EXAMPLE 111

### 1-methyl-N′-quinolin-5-ylcyclohexanecarbohydrazide

The product of Example 85A (464 mg, 2.00 mmol) was reacted with 1-methyl-cyclohexanecarboxylic acid (284 mg, 2.00 mmol) according to the procedure of Example 93 to provide 177 mg (31%) of the title compound as a white solid. MS (ESI) m/z 283.9 (M+H)⁺; NMR (CDCl₃) δ 1.23 (s, 3H), 1.25-1.34 (m, 3H), 1.40-1.52 (m, 5H), 2.04-2.09 (m, 2H), 6.71 (d, J=7.5 Hz, 1H), 7.37 (d, J=8.1 HZ, 1H), 7.44 (dd, J=8.6, 4.2 Hz, 1H), 7.53 (dd, J=7.5, 7.5 Hz, 1H), 8.35 (s, 1H), 8.70 (d, J=8.5 Hz, 1H), 8.84 (dd, J=4.1, 1.4 Hz, 1H), 9.69 (d, J-1.4 Hz, 1H).

### EXAMPLE 112

### 1,3-dimethyl-N′-quinolin-5-ylcyclohexanecarbohydrazide

The product of Example 85A (193 mg, 0.600 mmol) was reacted with 1,3-dimethyl-cyclohexanecarboxylic acid (78.1 mg, 0.500 mmol) according to the procedure as described in Example 93 to provide 18.0 mg (12%) of the title compound as a white solid. MS (ESI) m/z 297.9 (M+H)⁺; NMR (CDCl₃) δ 0.74-0.82 (m, 1H), 0.87 (d, J=6.4 Hz, 3H), 0.97-1.12 (s, 1H), 1.21 (s, 3H), 1.35-1.85 (m, 4H), 1.90 (s, 1H), 2.22-2.27 (m, 2H), 6.70 (d, J=7.5 Hz, 1H), 7.37 (d, J=8.1 Hz, 1H), 7.45 (dd, J=8.5, 4.1 HZ, 1H), 7.53 (dd, J=8.1, 8.0 Hz, 1H), 8.34 (d, J=1.7 Hz, 1H), 8.70 (d, J=8.5 Hz, 1H), 8.84 (dd, J=4.2, 1.5 Hz, 1H), 9.71 (d, J=2.0 Hz, 1H).

### EXAMPLE 113

### 1,3,3-trimethyl-N′-quinolin-5-ylcyclohexanecarbohydrazide

The product of Example 85A (193 mg, 0.600 mmol) was reacted with 1,3,3-trimethyl-cyclohexanecarboxylic acid (85.1 mg, 0.500 mmol) according to the procedure as described in Example 93 to provide 15.7 mg (10%) of the title compound as a white solid. MS (ESI) m/z 311.9 (M+H)⁺.

### EXAMPLE 114

### N′-quinolin-5-yloctahydronaphthalene-4a(2H)-carbohydrazide

The product of Example 85A (139 mg, 0.600 mmol) was reacted with octahydro-naphthalene-4a-carboxylic acid (91.1 mg, 0.500 mmol) according to the procedure as described in Example 93 to provide 9.1 mg (6%) of the title compound as a white solid. MS (LC-MS, APCI) m/z 323.5 (M+H)⁺; NMR (DMSO-d₆) δ 1.30-1.75 (m, 16H), 2.18-2.20 (m, 1H), 6.73 (d, J=7.5 Hz, 1H), 7.36 (d, J=8.1 Hz, 1H), 7.45 (dd, J=8.5, 4.4 Hz, 1H), 7.53 (dd, J=8.1, 8.0 Hz, 1H), 8.72 (d, J=8.5 Hz, 1H), 8.84 (dd, J=4.2, 1.5 Hz, 1H), 9.71 (d, J=2.0 Hz, 1H).

### EXAMPLE 115

### 2-phenyl-N′-quinolin-5-ylcyclohexanecarbohydrazide

The product of Example 85A (193 mg, 0.600 mmol) was reacted with 1,3,3-trimethyl-cyclohexanecarboxylic acid (85.1 mg, 0.500 mmol) according to the procedure as described in Example 93 to provide 14.1 mg (8.3%) of the title compound as a white solid. MS (ESI) m/z 346.1 (M+H)⁺; NMR (CDCl₃) δ 1.41-1.65 (m, 3H), 1.75-2.06 (m, 5H), 2.42-2.51 (m, 1H), 2.87-2.95 (m, 1H), 5.56 (d, J=7.5 Hz, 1H), 6.79 (br s, 1H), 7.10-7.19 (m, 2H), 7.26-7.30 M, 3H), 7.35-7.39 (m, 3H), 7.49 (d, J=8.5 Hz, 1H), 8.10 (d, J=8.5 Hz, 1H), 8.76 (d, J=4.4 Hz, 1H).

### EXAMPLE 116

### 2-[(2-methylphenoxy)methyl]-N′-quinolin-5-ylcyclohexanecarbohydrazide

The product of Example 85A (139 mg, 0.600 mmol) was reacted with 1,3,3-trimethyl-cyclohexanecarboxylic acid (126 mg, 0.500 mmol) according to the procedure as described in Example 93 to provide 18.2 mg (9%) of the title compound as a white solid. MS (ESI) m/z 390.1 (M+H)⁺; NMR (CDCl₃) δ 1.37-1.54 (m, 4H), 1.66-1.70 (m, 3H), 1.88-2.05 (m, 2H), 2.25 (s, 3H), 2.94-2.96 (m, 1H), 3.88-3.99 (m, 2H), 6.50 (D, J=7.5 Hz, 1H), 6.85-6.90 (m, 2H), 6.96 (dd, J=8.1, 8.0 Hz, 1H), 7.17-7.21 (m, 2H), 7.25 (d, J=8.5 Hz, 1H), 7.41 (dd, J=8.5, 4.1 Hz, 1H), 8.39 (d, J=2.0 Hz, 1H), 8.62 (d, J=8.5 HZ, 1H), 8.80 (dd, J-4.2, 1.5 Hz, 1H), 9.85 (D, J=2.4 Hz, 1H).

### EXAMPLE 117

### 2-methyl-4-oxo-N′-quinolin-5-yl-1,2,3,4-tetrahydronaphthalene-1-carbohydrazide

The product of Example 85A (139 mg, 0.600 mmol) was reacted with 2-methyl-4-oxo-1,2,3,4-tetrahydro-naphthalene-1-carboxylic acid (1.2 mg, 0.500 mmol) according to the procedure as described in Example 93 to provide 40.1 mg (23%) of the title compound as a white solid. MS (ESI) m/z 346.1 (M+H)⁺.

### EXAMPLE 118

### 2-methyl-N′-quinolin-5-ylbicyclo[2.2.1]hept-5-ene-2-carbohydrazide

The product of Example 85A (464 mg, 2.00 mmol) was reacted with 2-methyl-bicyclo[2.2.1]hept-5-ene-2-carboxylic acid (304 mg, 2.00 mmol) according to the procedure as described in Example 93 to provide 85.0 mg (14%) of the title compound as a white solid. MS (ESI) m/z 294.1 (M+H)⁺; NMR (CDCl₃) δ 0.77-0.81 (m, 1H), 1.21 (br s, 3H), 1.31-1.42 (m, 2H), 2.51-2.57 (m, 1HY), 2.82 (br s, 1H), 3.21 (br s, 1H), 6.15 (dd, J=5.4, 3.1 Hz, 1H), 6.29 (dd, J=5.6, 2.9 Hz, 1H), 6.68 (d, J=7.5 Hz, 1H), 7.37 (d, J=8.1 Hz, 1H), 7.45 (dd, J=8.6, 4.2 Hz, 1H), 7.53 (d, J=8.1, 8.0 Hz, 1H), 8.41 (d, J-2.0 Hz, 1H), 8.69 (d, J=7.8 Hz, 1H), 8.84 (dd, J=4.1 1.4 Hz, 1H), 9.89 (d, J=2.0 Hz, 1H).

### EXAMPLE 119

### 7,7-dimethyl-2-oxo-N′-quinolin-5-ylbicyclo[2.2.1]heptane-1-carbohydrazide

The product of Example 85A (139 mg, 0.600 mmol) was reacted with (S)-(+)-ketopinic acid (91.1 mg, 0.500 mmol) according to the procedure as described in Example 93 to provide 36.1 mg (22%) of the title compound as a white solid. MS (ESI) m/z 323.9 (M+H)⁺; NMR (DMSO-d₆) δ 1.09 (s, 3H), 1.13 (s, 4H), 1.43-1.51 (m, 1H), 1.85-2.09 (m, 3H), 2.31-2.41 (m, 1H), 2.50-2.58 (m, 1H), 6.98 (d, J=7.5 Hz, 1H), 7.38 (d, J=8.1 Hz, 1H), 7.44 (dd, J=8.6, 4.2 Hz, 1H), 7.54 (dd, J=8.1, 8.0 Hz, 1H), 8.52 (d, J=2.0 Hz, 1H), 8.69 (D, J=8.5 Hz, 1H), 8.83, (dd, J=4.1, 1.4 Hz, 1H), 9.49 (d, J=2.0 Hz, 1H).

### EXAMPLE 120

### 2-bicyclo[2.2.1]hept-2-yl-N′-quinolin-5-ylacetohydrazide

The product of Example 85A (139 mg, 0.600 mmol) was reacted with 2-norbornylacetic acid (77.1 mg, 0.500 mmol) according to the procedure as described in Example 93 to provide 38.0 mg (26%) of the title compound as a white solid. MS (ESI) m/z 296.1 (M+H)⁺; NMR (CDCl₃) δ 1.01-2.41 (m, 13H), 6.92 (d, J=6.4 Hz, 1H), 7.21-7.26 (m, 1H), 7.50 (dd, J=8.1, 8.0 Hz, 1H), 7.60 (d, J=8.4 Hz, 1H), 7.80 (br s, 1H), 8.31 (d, J=8.5 Hz, 1H), 8.80 (d, J=4.0 Hz, 1H).

### EXAMPLE 121

### 2-methyl-N′-quinolin-5-ylbicyclo[3.1.1]heptane-6-carbohydrazide

The product of Example 85A (139 mg, 0.600 mmol) was reacted with 2-norbornylacetic acid (77.1 mg, 0.500 mmol) according to the procedure as described in Example 93 to provide 8.8 mg (6%) of the title compound as a white solid. MS (ESI) m/z 296.1 (M+H)⁺.

### EXAMPLE 122

### 2,2-dicyclohexyl-N′-quinolin-5-ylacetohydrazide

The product of Example 85A (139 mg, 0.600 mmol) was reacted with dicyclohexyl-acetic acid (112 mg, 0.500 mmol) according to the procedure as described in Example 93 to provide 4.1 mg (2%) of the title compound as a white solid. MS (LCMS, APCI) m/z 366.3 (M+H)⁺.

### EXAMPLE 123

### 2-cyclohexyl-2-phenyl-N′-quinolin-5-ylacetohydrazide

The product of Example 85A (139 mg, 0.600 mmol) was reacted with cyclohexylphenylacetic acid (91.1 mg, 0.500 mmol) according to the procedure of Example 93 to provide 45.6 mg (25%) of the title compound as a white solid. MS (ESI) m/z 359.9 (M+H)⁺; NMR (DMSO-d₆) δ 0.72-0.84 (m, 1H), 1.11-1.29 (m, 5H), 1.58-1.61 (m, 2H), 1.74-1.78 (m, 1H), 1.90-1.93 (m, 1H), 1.99-2.07 (m, 1H), 6.46 (dd, J=7.1, 1.4 Hz, 1H),7.25-7.423 (m, 8H), 8.47 (s, 1H), 8.61 (d, J=8.5 Hz, 1H), 8.81 (dd, J=4.2, 1.5 Hz, 1H), 10.1 (dd, J=1.7 Hz, 1H).

### EXAMPLE 124

### 3-methyl-2-phenyl-N′-quinolin-5-ylbutanohydrazide

The product of Example 85A (139 mg, 0.600 mmol) was reacted with 3-methyl-2-phenyl-butyric acid (83.1 mg, 0.500 mmol) according to the procedure as described in Example 93 to provide 30.8 mg (19%) of the title compound as a white solid. MS (ESI) m/z 319.9 (M+H)⁺; NMR (DMSO-d₆) δ 0.69 (d, J=6.8 Hz, 3H), 1.09 (d, J-6.4 Hz, 3H), 2.31-2.40 (m, 1H), 3.20 (d, J=10.8 Hz, 1H), 6.45 (dd, J=7.0, 1.5 Hz, 1H), 7.25-7.43 (m, 8H), 8.47 (d, J=1.7 Hz, 1H), 8.62 (d, J=8.8 Hz, 1H), 8.81 (dd, =4.2, 1.5 Hz, 1H), 10.1 (d, J=2.0 Hz, 1H).

### EXAMPLE 125

### 2-(4-cyclohexylphenyl)-3-methyl-N′-quinolin-5-ylbutanohydrazide

The product of Example 85A (139 mg, 0.600 mmol) was reacted with 2-(4-cyclohexyl-phenyl)-3-methyl-butyric acid (130 mg, 0.500 mmol) according to the procedure as described in Example 93 to provide 27.8 mg (14%) of the title compound as a white solid. MS (ESI) m/z 402.2 (M+H)⁺; NMR (DMSO-d₆) δ 0.70 (d, J=6.8 Hz, 3H), 1.08 (d, J=6.4 Hz, 3H), 1.21-1.43 (m, 5H), 1.69-1.81 (m, 5H), 2.26-2.37 (m, 1H), 3.16 (d, J=10.8 Hz, 1H), 6.45 (dd, J=6.4, 2.0 Hz, 1H), 7.19 (d, J=8.5 Hz, 2H), 7.29-7.42 (m, 5H), 8.46 (d, J=2.0 Hz, 1H), 8.60 (dd, J=8.5, 1.4 Hz, 1H), 8.81 (dd, J=4.1, 1.7 Hz, 1H), 10.0 (d, J=2.0 Hz, 1H).

### EXAMPLE 126

### 2-[1-(4-chlorophenyl)cyclobutyl]-2-methyl-N′-quinolin-5-ylpropanohydrazide

The product of Example 85A (139 mg, 0.600 mmol) was reacted with 2-norbornylacetic acid (126 mg, 0.500 mmol) according to the procedure as described in Example 93 to provide 13.4 mg (7%) of the title compound as a white solid. MS (APCI) m/z 393.4 (M+H)⁺; NMR (CDCl₃) δ 1.35 (S, 6H), 1.72-1.88 (m, 2H), 2.33-2.43 (m, 2H), 2.77-2.86 (m, 2H), 6.5 (d, J=7.5 Hz, 1H), 6.78 (d, J=4.4 Hz, 1H), 7.01 (d, J=4.1 Hz, 1H), 7.17-7.25 (m, 4H), 7.37 (dd, J=8.6, 4.2 Hz, 1H), 7.50 (dd, J=8.1, 8.0 Hz, 1H), 7.70 (d, J=8.5 HZ, 1H), 8.27 (d, J=8.5 Hz, 1H), 8.89 (D, J=3.0 Hz, 1H).

### EXAMPLE 127

### 2-methoxy-2-(1-naphthyl)-N′-quinolin-5-ylpropanohydrazide

The product of Example 85A (139 mg, 0.600 mmol) was reacted with 2-methoxy-2-naphthalen-1-yl-propionic acid (115 mg, 0.500 mmol) according to the procedure as described in Example 93 to provide 19.0 mg (10%) of the title compound as a white solid. MS (ESI) m/z 372.1 (M+H)⁺; NMR (DMSO-d₆) δ 2.01 (s, 3H), 3.07 (s, 3H), 6.79 (d, J=7.5 Hz, 1H), 7.34-7.57 (m, 6H), 7.68 (d, J=6.4 Hz, 1H), 7.91-7.98 (m, 2H), 8.42 (s. 1H), 8.63-8.69 (m, 2H), 8.81 (dd, J=4.1, 1.4 Hz, 1H), 10.3 (s, 1H).

### EXAMPLE 128

### 2,3-diphenyl-N′-quinolin-5-ylpropanohydrazide

The product of Example 85A (139 mg, 0.600 mmol) was reacted with 2,3-diphenyl-propionic acid (113 mg, 0.500 mmol) according to the procedure as described in Example 92 to provide 36.2 mg (20%) of the title compound as a white solid. MS (ESI) m/z 367.9 (M+H)⁺; NMR (DMSO-d₆) δ 2.99 (dd, J-13.0, 4.9 Hz, 1H), 3.39 (dd, J=13.4, 10.7 Hz, 1H), 4.03 (dd, J=10.5, 5.1 Hz, 1H), 5.77 (d, J=7.5 HZ, 1H), 7.13-7.19 (m, 2H), 7.27-7.39 (m, 9H), 7.51 (d, J=7.1 Hz, 2H), 8.40 (br s, 1H), 8.53 (dd, J=8.8 Hz, 1H), 8.79 (dd, J=4.2, 1.5 Hz, 1H), 9.97 (br s, 1H).

### e) METHODS OF THE INVENTION

Compounds and compositions of the invention are useful for modulating the effects of P2X₇ receptor activation. In particular, the compounds and compositions of the invention 5 can be used for treating and preventing disorders modulated by P2X₇ receptors. Typically, such disorders can be ameliorated by selectively inhibiting or antagonizing P2X₇ receptors in a mammal, preferably by administering a compound or composition of the invention, either alone or in combination with another active agent, for example, as part of a therapeutic regimen.

The compounds of the invention, including but not limited to those specified in the examples, possess an affinity for P2X₇ receptors. As P2X₇ receptor antagonists, the compounds of the invention can be useful for the treatment and prevention of a number of P2X₇ receptor-mediated diseases or conditions.

For example, on glial cells, the P2X₇ receptor has been shown to mediate release of glutamate (Anderson C. et al. Drug Dev. Res. Vol. 50. page 92, 2000). Upregulation of the P2X₇ receptor, most likely on activated microglia, was reported in association with ischemic damage and necrosis induced by occlusion of middle cerebral artery in rat brain (Collo G. et al. Neuropharmacology, Vol. 36, pages 1277-1283, 1997). Recent studies indicate a role of the P2X₇ receptor in the generation of superoxide in microglia, and upregulation of P2X₇ receptors around β-amyloid plaques in a transgenic mouse model for Alzheimer's disease (Parvathenani et al., J. Biol. Chemistry, Vol. 278, pages 13300-13317, 2003) and in multiple sclerosis lesions from autopsy brain sections (Narcisse et al., Glia, Vol. 49, pages 245-258 (2005). As such, P2X₇ receptor antagonists are suitable for the prevention, treatment or amelioration of degenerative states including, but not limited to for example, damage induced ischemia, depression, Alzheimer's disease (AD), multiple sclerosis.

Oxidized ATP (oATP), a nonselective and irreversible P2X₇ antagonist, was recently reported to possess peripherally mediated antinociceptive properties in inflamed rats (Dell'Antonio et al. Neuroscience Lett., Vol. 327, pages 87-90, 2002). Activation of P2X₇ receptors localized on presynaptic terminals in the central and peripheral nervous systems (Deuchars et al J. Neuroscience, Vol. 21, pages 7143-7152,2001) induced release of the excitatory amino acid neurotransmitter glutamate. A link between a P2X₇ purinoceptor gene and chronic, inflammatory and neuropathic pain has also been reported (Hatcher et al., The 6th International Conference on the Mechanisms and Treatment of Neuropathic Pain. San Francisco, Calif.—Sep. 18-20, 2003).

As such P2X₇ receptor antagonists are suitable for the prevention, treatment or amelioration of pain in general, more particularly of neuropathic pain, thermal hyperalgesia, allodinya, and inflammatory pain. Representative compounds of the present invention were active in reducing tactile allodynia when tested using the Ching Model and the CFA Model (see Biological Activity section).

Antagonists to the P2X₇ receptor significantly improved functional recovery and decreased cell death in spinal cord injury (SCI) animal models. Rats with SCI were administered P2X₇ receptor irreversible antagonists oATP and PPADS with a resulting decrease of histological injury and improved recovery of motor function after the lesions (Wang et al., Nature Medicine Vol. 10, pages B21-B27, 2004). These facts indicate that as such P2X₇ receptor antagonists are suitable for promoting neuroregeneration and neurorecovery in central and peripheral tissues after, for example, spinal cord injury.

### f) BIOLOGICAL ACTIVITY

In Vitro Data

Tissue Culture: Cells of the THP-1 monocytic cell line (American Type Culture Collection, Rockville, Md.) were maintained in the log phase of growth in RPMI medium containing high glucose and 10% fetal calf serum (BRL, Grand Island, N.Y.) according to established procedures (Humphrey and Dubyak, J. Immunol. Vol. 275, pages 26792-26798, 1996). Fresh vials of frozen THP-1 cells were initiated for growth every eight weeks. To differentiate THP-1 cells into a macrophage phenotype, a final concentration of 25 ng/ml of LPS and 10 ng/ml of IFNγ were added to the cells (Humphrey and Dubyak 1996) either for 3 hours for IL-1β release assays or overnight (16 hours) for pore formation studies. 1321N1 cells stably expressing the recombinant human P2X₇ receptor were grown and used according to previously published protocols (Bianchi, et al, Eur. J. Pharmacol. Vol. 376, pages 127-138, 1999; Lynch et al., Mol. Pharmacol. Vol. 56, pages 1171-1181, 1999). For both the pore formation and IL-1β release assays, cell density and viability were routinely assessed prior to each experiment by trypan dye exclusion and cells found to be >90% viable following differentiation.

IL-1β Release: THP-1 cells were plated in 24-well plates at a density of 1×10⁶ cells/well/ml. On the day of the experiment, cells were differentiated with 25 ng/ml LPS and 10 ng/ml final concentration of γIFN for 3 hours at 37° C. Solutions of antagonist compounds were prepared by serial dilutions of a 10 mM DMSO solution of the antagonist into the PBS solution. In the presence of the differentiation media, the cells were incubated with the antagonists of the present invention for 30 minutes at 37° C. followed by a challenge with 1 mM BzATP for an additional 30 minutes at 37° C. Supernatants of the samples were collected after a 5 minute centrifugation in microfuge tubes to pellet the cells and debris and to test for mature IL-1β released into the supernatant using either R & D Systems Human IL-1β ELISA assay or Endogen Human IL-1β ELISA, following the manufacturer's instructions. The maximum IL-1β release at each concentration of test compound was normalized to that induced by BzATP alone to determine the activity of the test compound. Antagonist potency was expressed as the concentration producing a 50% reduction in release of IL-1β or IC₅₀. Representative compounds of the present invention when tested with the above assay demonstrated antagonist activity at the P2X₇ receptor with IC₅₀ equal or less than 10 μM, preferably less than 0.5 μM, and most preferably less than 0.5 μM.

In Vivo Data—Determination of Antinociceptive Effect

Animal handling and experimental protocols were approved by the Institutional Animal Care and Use Committee (IACUC) at Abbott Laboratories. For all surgical procedures, animals were maintained under halothane anesthesia (4% to induce, 2% to maintain), and the incision sites were sterilized using a 10% povidone-iodine solution prior to and after surgeries.

CFA model: The capacity of the antagonists to reduce inflammatory hyperalgesia was evaluated using the complete Freund's adjuvant (CFA) model. In these experiments, animals were subjected to intraplantar injection of CFA 48 hours before administration of the P₂X₇ antagonists. Inhibition of thermal hyperalgesia was determined 30 minutes after antagonist administration by observation of paw withdrawal latency and comparison to response of the contralateral paw. Representative compounds were active in reducing tactile allodynia when tested using this model.

Chung model: Efficacy in the reduction of neuropathic pain was evaluated using the L5/L6 spinal nerve tight ligation (Chung) model in rats. In these experiments, spinal nerve ligation was performed 7-14 days prior to assay. Tactile allodynia was induced by application of a von Frey hair 30 minutes after administration of the antagonist. Reduction in tactile allodynia was measured by determination of the paw withdrawal threshold and comparison to the contralateral paw. Representative compounds were active in reducing tactile allodynia when tested using this model. (Jarvis et al., Proc. Natl. Acad. USA Vol. 99, pages 17179-17184, 2002).

Zymosan Method: Mice were dosed with experimental compounds orally or subcutaneously 30 minutes prior to injection of zymosan. Mice were then injected intraperitonealy with 2 mg/animal of zymosan suspended in saline. Four hours later the animals were euthanized by CO₂ inhalation and the peritoneal cavities lavaged with 2×1.5 mL of ice cold phosphate buffered saline containing 10 units of heparin/ml. For IL-1β determination the samples were spun at 10,000×g in a refrigerated microfuge (4° C.), supernatants removed and frozen until ELISAs (Enzyme Linked Immuno-Assay) were performed. ELISAs were performed according to manufacture's instructions. IL-1β was determined relative to vehicle control (Perretti M. et al., Agents Actions Vol 35(1-2) pages 71-78 (1992); Torok K, et al., Inflamm Res. Vol 44(6) pages 248-252 (1995)). Representative compounds of this invention were active as P2X7 antagonists in inhibiting IL-1β release in this assay.

Using these methods, representative compounds had ED₅₀ equal or lower than 500 μmol/kg, preferably less than 50 μmol/kg.

What is claimed is:

1. A compound of formula (I) 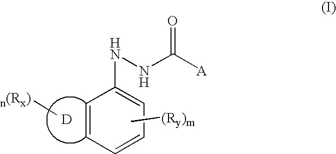
or a pharmaceutically acceptable salt or prodrug thereof, wherein
D is a five or six-membered heteroaryl ring selected from the group consisting of pyridine, pyridizine, pyrimidine, pyrazine, pyrazole, isothiazole, thiazole, isoxazole, oxazole and furazan;
m is 0, 1, 2 or 3;
n is 0, 1, 2, 3 or 4;
Rₓ and R_{y} are independently selected from the group consisting of alkyl, alkenyl, halogen, nitro, cyano, haloalkyl, —C(O)alkyl, —C(O)OH, —C(O)Oalkyl, —C(O)NH₂, —C(O)N(H)(alkyl), —C(O)N(alkyl)₂ and -G₁-G₂-G₃;
G₁ at each occurrence is independently selected from the group consisting of a bond O, S and —N(R₁₀₁)—;
G₂ at each occurrence is independently selected from the group consisting of a bond, alkyl and -alkyl-N(R₁₀₁)-alkyl-;
G₃ at each occurrence is independently selected from the group consisting of hydrogen, alkyl, —N(R₁₀₂)(R₁₀₃), and —O(R₁₀₂);
R₁₀₁ at each occurrence is independently selected from the group consisting of hydrogen, alkyl, alkenyl, haloalkyl, haloalkenyl, hydroxyalkyl, and alkoxyalkyl;
R₁₀₂ at each occurrence is independently selected from the group consisting of hydrogen alkyl and haloalkyl;
R₁₀₃ at each occurrence is selected from the group consisting of hydrogen, alkyl, alkenyl, haloalkyl, hydroxyalkyl, alkxoyalkyl, -alkyl-NH₂, -alkyl-N(H)(alkyl), -alkyl-N(alkyl)₂, —C(O)alkyl, and -alkyl-C(O)O(alkyl);
alternatively, R₁₀₂ and R₁₀₃, together with the nitrogen atom to which they are attached, form a saturated four to nine membered heterocyclic ring; wherein the heterocyclic ring may comprise a second ring heteroatom selected from the group consisting of nitrogen and oxygen, and the ring is substituted with 0, 1, 2 or 3 substituents selected from the group consisting of —OH, halogen, alkyl, alkenyl, hydroxyalkyl, -alkyl-NH₂, -alkyl-N(H)(alkyl), -alkyl-N(alkyl)₂, and —N(H)(—CH₂CH₂OH);
A is R₁ or -L₁-R₂;
L₁ is C₁-C₆ alkylenyl substituted with 0, 1 or 2 substituents selected from the group consisting of alkoxy, halogen, haloalkyl, and R_{c};
R₁ is selected from the group consisting of cycloalkenyl, cycloalkyl and heterocycle; wherein each RI is independently substituted with 0, 1, 2, 3, 4 or 5 substituents independently selected from the group consisting of alkenyl, alkyl, alkynyl, halogen, haloalkyl, nitro, oxo, R_{c}, -alkylR_{c}, -alkylOR_{c} and -G₁-G₂-G₃;
R₂ is selected from the group consisting of heteroaryl, aryl, cycloalkenyl and cycloalkyl; wherein each R₂ is independently substituted with 0, 1 or 2 substituents independently selected from the group consisting of alkyl, haloalkyl, -G₁-G₂-G₃ and R_{c}; and
R_{c} at each occurrence is independently selected from the group consisting of cycloalkyl, cycloalkenyl, heterocycle, aryl and hetroaryl; wherein each R_{c} is independently substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)OH, —C(O)Oalkyl, —C(O)NH₂, —C(O)N(H)(alkyl) and —C(O)N(alkyl)₂.

2. The compound of formula I according to claim 1 wherein D is pyridine.

3. The compound of claim 2, wherein said compound of formula (I) is selected from the group consisting of

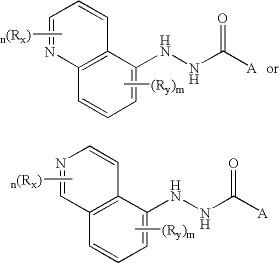

4. The compound of claim 3, wherein the compound has formula 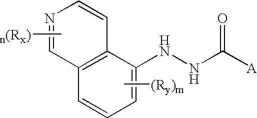
n is 0;
m is 0

5. The compound of claim 4, wherin A is -L₁-R₂.

6. The compound of claim 4, wherein A is R₁.

7. The compound of claim 6, wherein R₁ is cycloalkyl.

8. The compound of claim 7, a therapeutically acceptable salt, solvate, prodrug, or salt of a prodrug thereof, wherein the compound is
N′-isoquinolin-5-yladamantane-1-carbohydrazide.

9. The compound of claim 3, wherein the compound has formula

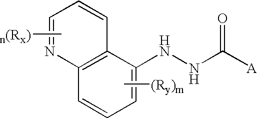

10. The compound of claim 9, wherein A is R₁.

11. The compound of claim 10, wherein R₁ is a monocyclic cycloalkyl.

12. The compound of claim 11, a therapeutically acceptable salt, solvate, prodrug, or salt of a prodrug thereof, wherein the compound is selected form the group consisting of
1-methyl-2,2-diphenyl-N′-quinolin-5-ylcyclopropanecarbohydrazide;
2,2,3,3-tetramethyl-N′-quinolin-5-ylcyclopropanecarbohydrazide;
1-phenyl-N′-quinolin-5-ylcyclopropanecarbohydrazide;
N′-quinolin-5-yl-1-thien-2-ylcyclopropanecarbohydrazide;
1-cyclohexyl-N′-quinolin-5-ylcyclopropanecarbohydrazide;
1-benzyl-N′-quinolin-5-ylcyclopentanecarbohydrazide;
1-(2-fluorophenyl)-N′-quinolin-5-ylcyclohexanecarbohydrazide;
1-(3-fluorophenyl)-N′-quinolin-5-ylcyclohexanecarbohydrazide;
1-(4-fluorophenyl)-N′-quinolin-5-ylcyclohexanecarbohydrazide;
1-(4-methoxyphenyl)-N′-quinolin-5-ylcyclohexanecarbohydrazide;
3-isopropyl-1-methyl-N′-quinolin-5-ylcyclopentanecarbohydrazide;
(1R,3S)-1,2,2,3-tetramethyl-N′-quinolin-5-ylcyclopentanecarbohydrazide;
1-methyl-N′-quinolin-5-ylcyclohexanecarbohydrazide;
1,3-dimethyl-N′-quinolin-5-ylcyclohexanecarbohydrazide;
1,3,3-trimethyl-N′-quinolin-5-ylcyclohexanecarbohydrazide;
2-phenyl-N′-quinolin-5-ylcyclohexanecarbohydrazide; and
2-[(2-methylphenoxy)methyl]-N′-quinolin-5-ylcyclohexanecarbohydrazide.

13. The compound of claim 11, wherein the monocyclic cycloalkyl contains one or two bridges.

14. The compound of claim 13, a therapeutically acceptable salt, solvate, prodrug, or salt of a prodrug thereof, wherein the compound is selected form the group consisting of
N′-quinolin-5-yladamantane-1-carbohydrazide;
N′-(2-chloroquinolin-5-yl)adamantane-1-carbohydrazide;
N′-quinolin-1-ylhexahydro-2,5-methanopentalene-3a(1H)-carbohydrazide;
3-chloro-N′-quinolin-5-yladamantane-1-carbohydrazide;
3-bromo-N′-quinolin-5-yladamantane-1-carbohydrazide;
3-ethyl-N′-quinolin-5-yladamantane-1-carbohydrazide;
3,5-dimethyl-N′-quinolin-5-yladamantane-1-carbohydrazide;
3-(1,1,2,3,3,3-hexafluoropropyl)-N′-quinolin-5-yladamantane-1-carbohydrazide;
2-methyl-N′-quinolin-5-ylbicyclo[2.2.1]hept-5-ene-2-carbohydrazide;
7,7-dimethyl-2-oxo-N′-quinolin-5-ylbicyclo[2.2.1]heptane-1-carbohydrazide; and
2-methyl-N′-quinolin-5-ylbicyclo[3.1.1]heptane-6-carbohydrazide.

15. The compound of claim 10, wherein R₁ is a bicyclic cycloalkyl.

16. The compound of claim 15, a therapeutically acceptable salt, solvate, prodrug, or salt of a prodrug thereof, wherein the compound is selected form the group consisting of
N′-quinolin-5-ylspiro[2.5]octane-1-carbohydrazide; and
1-methyl-N′-quinolin-5-ylindane-2-carbohydrazide
N′-quinolin-5-yloctahydronaphthalene-4a(2H)-carbohydrazide.
2-methyl-4-oxo-N′-quinolin-5-yl-1,2,3,4-tetrahydronaphthalene-1-carbohydrazide

17. The compound of claim 10, wherein R₁ is a tricyclic cycloalkyl.

18. The compound of claim 17, a therapeutically acceptable salt, solvate, prodrug, or salt of a prodrug thereof, wherein the compound is
N′-quinolin-5-yldodecahydro-1H-fluorene-9-carbohydrazide.

19. The compound of claim 9, wherein A is -L₁-R₂.

20. The compound of claim 19, wherein R₂ is cycloalkyl.

21. The compound of claim 20, a therapeutically acceptable salt, solvate, prodrug, or salt of a prodrug thereof, wherein the compound is selected form the group consisting of
2-(1-adamantyl)-N′-quinolin-5-ylacetohydrazide;
3-(1-adamantyl)-N′-quinolin-5-ylpropanohydrazide;
2-bicyclo[2.2.1]hept-2-yl-N′-quinolin-5-ylacetohydrazide;
2,2-dicyclohexyl-N′-quinolin-5-ylacetohydrazide;
2-cyclohexyl-2-phenyl-N′-quinolin-5-ylacetohydrazide;
3-methyl-2-phenyl-N′-quinolin-5-ylbutanohydrazide;
2-(4-cyclohexylphenyl)-3-methyl-N′-quinolin-5-ylbutanohydrazide;
2-[1-(4-chlorophenyl)cyclobutyl]-2-methyl-N′-quinolin-5-ylpropanohydrazide;
2-methoxy-2-(1-naphthyl)-N′-quinolin-5-ylpropanohydrazide; and
2,3-diphenyl-N′-quinolin-5-ylpropanohydrazide.

22. A pharmaceutical composition comprising a therapeutically effective amount of a compound of formula I according to claim 1, or a therapeutically acceptable salt, solvate, prodrug, salt of a prodrug, or combination thereof, and a pharmaceutically acceptable carrier.

23. A method for inhibiting P2X₇ activity comprising administering to a patient in need of such treatment a therapeutically effective amount of a compound of formula I according to claim 1, or a therapeutically acceptable salt, solvate, prodrug, salt of a prodrug, or combination thereof.

24. A method for treating a disorder selected from the group consisting of chronic inflammatory pain, neuropathic pain, inflammation, neurodegeneration, depression and promoting neuroregeneration, comprising administering to a patient in need of such treatment a pharmaceutical composition of claim 22.

25. A method for treating inflammation comprising administering to a patient in need of such treatment a therapeutically effective amount of a compound of formula I according to claim 1, or a therapeutically acceptable salt, solvate, prodrug, salt of a prodrug, or combination thereof.

26. A method for treating neurodegeneration comprising administering to a patient in need of such treatment a therapeutically effective amount of a compound of formula I according to claim 1, or a therapeutically acceptable salt, solvate, prodrug, salt of a prodrug, or combination thereof.

27. A method for treating neuropathic pain comprising administering to a patient in need of such treatment a therapeutically effective amount of a compound of formula I according to claim 1, or a therapeutically acceptable salt, solvate, prodrug, salt of a prodrug, or combination thereof.

28. A method for treating chronic inflammatory pain comprising administering to a patient in need of such treatment a therapeutically effective amount of a compound of formula I according to claim 1, or a therapeutically acceptable salt, solvate, prodrug, salt of a prodrug, or combination thereof.

29. A method for promoting neuroregeneration comprising administering to a patient in need of such treatment a therapeutically effective amount of a compound of formula I according to claim 1, or a therapeutically acceptable salt, solvate, prodrug, salt of a prodrug, or combination thereof.

30. A method for treating depression comprising administering to a patient in need of such treatment a therapeutically effective amount of a compound of formula I according to claim 1, or a therapeutically acceptable salt, solvate, prodrug, salt of a prodrug, or combination thereof.

31. A method for treating pain, neuropathic pain, inflammation, chronic inflammatory pain, neurodegeneration, depression and promoting neuroregeneration in a mammal in need comprising administering to said mammal in need of such treatment a therapeutically effective amount of a compound of formula (II), 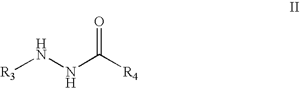
a pharmaceutically acceptable salt, ester, amide or prodrug thereof, wherein
R₃ is selected from the group consisting of alkyl, cycloalkyl, cycloalkenyl, heterocyclealkyl, aryl, and heteroaryl; wherein the cycloalkyl, cycloalkenyl, heterocyclealkyl, aryl and heteroaryl are independently substituted with 0, 1, 2, 3, 4 or 5 substituents independently selected from the group consisting of alkyl, alkenyl, halogen, nitro, cyano, haloalkyl, -G₁-G₂-G₃, —C(O)alkyl, —C(O)OH and —C(O)Oalkyl;
R₄ is selected from the group consisting of alkyl, alkenyl, alkynyl, cycloalkenyl, cycloalkyl and heterocycle; wherein the alkyl is substituted with 0, 1 or 2 substituents independently selected from the group consisting of Rₐ and R_{b}, and wherein each of the cycloalkenyl, cycloalkyl and heterocycle is independently substituted with 0, 1, 2, 3, 4, or 5 substituents independently selected from the group consisting of alkenyl, alkyl, alkynyl, halogen, haloalkyl, nitro, oxo, aryloxy, -G₁-G₂-G₃, —S(O)₂alkyl, —C(O)alkyl, R_{b}, -alkylR_{b}, and -alkylOR_{b}; wherein the aryl moiety of the aryloxy is substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)OH, —C(O)Oalkyl, —C(O)NH₂, —C(O)N(H)(alkyl) and —C(O)N(alkyl)₂;
Rₐ at each occurrence is independently selected from the group consisting of —OH, alkoxy, —OR_{b}, —O-alkyl-R_{b}, —S(alkyl), —SR_{b}, —S(O)₂alkyl, —S(O)₂R_{b}, —C(O)alkyl, —C(O)R_{b}, —N(H)C(O)alkyl, —N(H)C(O)R_{b}, —N(H)S(O)₂alkyl, —N(H)S(O)₂R_{b}, —C(O)N(H)alkyl and —C(O)N(H)R_{b};
R_{b} at each occurrence is independently selected from the group consisting of cycloalkyl, cycloalkenyl, heterocycle, aryl and hetroaryl; wherein each R_{b} at each occurrence is independently substituted with 0, 1, 2, 3, 4 or 5 substituents independently selected from the group consisting of alkyl, alkenyl, halogen, nitro, cyano, haloalkyl, —OH, alkoxy, aryloxy, haloalkoxy, —S(O)₂alkyl, —NH₂, —N(H)(alkyl), —N(alkyl)₂, —N(H)S(O)₂alkyl, —N(alkyl)S(O)₂alkyl, —N(H)C(O)alkyl, —N(alkyl)C(O)alkyl, —C(O)alkyl, —C(O)NH₂, —C(O)N(H)(alkyl), —C(O)N(alkyl)₂, —C(O)OH and —C(O)Oalkyl; wherein the aryl moiety of the aryloxy is substituted with 0, 1, 2, 3, 4 or 5 substituents independently selected from the group consisting of alkyl, alkenyl, halogen, nitro, cyano, haloalkyl, —OH, alkoxy, haloalkoxy, —NH₂, —N(H)(alkyl), —N(alkyl)₂, —C(O)alkyl, —C(O)NH₂, —C(O)N(H)(alkyl), —C(O)N(alkyl)₂, —C(O)OH and —C(O)Oalkyl;
G₁ at each occurrence is independently selected from the group consisting of a bond O, S and —N(R₁₀₁)—;
G₂ at each occurrence is independently selected from the group consisting of a bond, alkyl and -alkyl-N(R₁₀₁)-alkyl-;
G₃ at each occurrence is independently selected from the group consisting of hydrogen, alkyl, —N(R₁₀₂)(R₁₀₃), and —O(R₁₀₂);
R₁₀₁ at each occurrence is independently selected from the group consisting of hydrogen, alkyl, alkenyl, haloalkyl, haloalkenyl, hydroxyalkyl, and alkoxyalkyl;
R₁₀₂ at each occurrence is independently selected from the group consisting of hydrogen alkyl and haloalkyl; and
R₁₀₃ at each occurrence is independently selected from the group consisting of hydrogen, alkyl, alkenyl, haloalkyl, hydroxyalkyl, alkxoyalkyl, -alkyl-NH₂, -alkyl-N(H)(alkyl), -alkyl-N(alkyl)₂, —C(O)alkyl, and -alkyl-C(O)O(alkyl);
alternatively, R₁₀₂ and R₁₀₃, together with the nitrogen atom to which they are attached, form a saturated four to nine membered heterocyclic ring; wherein the heterocyclic ring may comprise a second ring heteroatom selected from the group consisting of nitrogen and oxygen, and the ring is substituted with 0, 1, 2 or 3 substituents selected from the group consisting of —OH, halogen, alkyl, alkenyl, hydroxyalkyl, -alkyl-NH₂, -alkyl-N(H)(alkyl), -alkyl-N(alkyl)₂, and —N(H)(—CH₂CH₂OH).

32. The method according to claim 31, wherein R₃ is aryl and aryl is phenyl.

33. The method according to claim 32, wherein R₄ is alkyl.

34. The method according to claim 33, wherein the compound is selected from the group consisting of
N′-(2-methylphenyl)butanohydrazide;
N′-(2-methylphenyl)pentanohydrazide;
3-methyl-N′-(2-methylphenyl)butanohydrazide;
2,2-dimethyl-N′-(2-methylphenyl)propanohydrazide;
N′-(2-methylphenyl)hexanohydrazide;
2-methyl-N′-(2-methylphenyl)pentanohydrazide;
3-methyl-N′-(2-methylphenyl)pentanohydrazide;
4-methyl-N′-(2-methylphenyl)pentanohydrazide;
2,2-dimethyl-N′-(2-methylphenyl)butanohydrazide;
3,3-dimethyl-N′-(2-methylphenyl)butanohydrazide;
2-ethyl-N′-(2-methylphenyl)butanohydrazide;
N′-(2-methylphenyl)heptanohydrazide;
2-methyl-N′-(2-methylphenyl)propanohydrazide; and
N′-(2-methylphenyl)pent-4-ynohydrazide.

35. The method according to claim 31, wherein R₃ is phenyl, R₄ is alkyl,
wherein alkyl is substituted with 1 or 2 Rₐ, wherein Rₐ is independently selected from the group consisting of —OH, alkoxy, —OR_{b}, —O-alkyl-R_{b}, —S(alkyl), —SR_{b}, —S(O)₂alkyl, —S(O)₂R_{b}, —C(O)alkyl, —C(O)R_{b}, —N(H)C(O)alkyl, —N(H)C(O)R_{b}, —N(H)S(O)₂alkyl, —N(H)S(O)₂R_{b}, —C(O)N(H)alkyl and —C(O)N(H)R_{b}; wherein R_{b} at each occurrence is independently selected from the group consisting of cycloalkyl, cycloalkenyl, heterocycle, aryl and heteroaryl.

36. The method of claim 35, wherein the compound is selected from the group consisting of
3-ethoxy-N′-(2-methylphenyl)propanohydrazide;
N′-(2-methylphenyl)-2-((2S)-acetylamino)-4-methylpentanohydrazide;
N′-(2-methylphenyl)-2-(methylthio)acetohydrazide.
N′-(2-methylphenyl)-3-phenoxypropanohydrazide;
N′-(2-methylphenyl)-2-((furan-2-yl)carbonylamino)acetohydrazide;
N′-(2-methylphenyl)-2-(pyrimidin-2-ylthio)acetohydrazide;
N′-(2-methylphenyl)-4-oxo-4-phenyl-3-azabutanohydrazide;
N′-(2-methylphenyl)-4-phenoxybutanohydrazide;
N′-(2-methylphenyl)-4-oxo-4-thien-2-ylbutanohydrazide;
5-[2-(2-methylphenyl)hydrazino]-5-oxo-N-phenylpentanamide;
4-(4-methoxyphenyl)-N′-(2-methylphenyl)-4-oxobutanohydrazide;
N′-(2-methylphenyl)-3-(phenylsulfonyl)propanohydrazide;
4-methyl-N-{5-[2-(2-methylphenyl)hydrazino]-5-oxomethyl}benzenesulfonamide;
N′-(2-methylphenyl)-2-(methylthio)acetohydrazide;
2-(benzyloxy)-N′-(2-methylphenyl)acetohydrazide;
2-(3-methylphenoxy)-N′-(2-methylphenyl)acetohydrazide;
2-(2-methylphenoxy)-N′-(2-methylphenyl)acetohydrazide; and
2-(4-methylphenoxy)-N′-(2-methylphenyl)acetohydrazide.

37. The method according to claim 31, wherein R₃ is phenyl, R₄ is alkyl,
wherein alkyl is substituted with 2 groups selected from the group consisiting of Rₐ and R_{b}.

38. The method according to claim 31, wherein the compound is selected from the group consisiting of
(2R)-2-methoxy-N′-(2-methylphenyl)-2-phenylacetohydrazide, and
(2S)-2-methoxy-N′-(2-methylphenyl)-2-phenylacetohydrazide.

39. The method of claim 31, R₃ is phenyl, R₄ is alkyl, wherein alkyl is substituted with R_{b},
wherein R_{b} is independently selected from the group consisting of cycloalkyl, cycloalkenyl, heterocycle, aryl and heteroaryl.

40. The method of claim 39 wherein the compound is selected form the group consisting of
2-cyclopentyl-N′-(2-methylphenyl)acetohydrazide;
2-cyclohexyl-N′-(2-methylphenyl)acetohydrazide;
2-(1-adamantyl)-N′-(2-methylphenyl)acetohydrazide;
N′-(2-methylphenyl)-3-phenylpropanohydrazide;
(2S)-N′-(2-methylphenyl)-2-phenylbutanohydrazide;
N′-(2-methylphenyl)-4-phenylbutanohydrazide;
N′-(2-methylphenyl)-4-thien-2-ylbutanohydrazide;
2-(3,5-difluorophenyl)-N′-(2-methylphenyl)acetohydrazide;
3-(3-methoxyphenyl)-N′-(2-methylphenyl)propanohydrazide;
3-(4-methoxyphenyl)-N′-(2-methylphenyl)propanohydrazide;
(2R)-2-hydroxy-N′-(2-methylphenyl)-4-phenylbutanohydrazide;
3-(2-chlorophenyl)-N′-(2-methylphenyl)propanohydrazide;
3-(4-chlorophenyl)-N′-(2-methylphenyl)propanohydrazide;
3-methyl-N′-(2-methylphenyl)-2-phenylpentanohydrazide;
N′-(2-methylphenyl)-2,2-diphenylacetohydrazide;
N′-(2-methylphenyl)-2-[4-(methylsulfonyl)phenyl]acetohydrazide;
N′-(2-methylphenyl)-2-(3-phenoxyphenyl)acetohydrazide; and
2-bicyclo[2.2.1]hept-2-yl-N′-(2-methylphenyl)acetohydrazide.

41. The method according to claim 31, R₃ is phenyl, and R₄ is cycloalkyl.

42. The method of claim 41, wherein the compound is selected form the group consisting of
N′-(2-methylphenyl)adamantane-1-carbohydrazide;
N′-(2-methylphenyl)-4-pentylbicyclo[2.2.2]octane-1-carbohydrazide;
N′-(2-methylphenyl)-1-phenylcyclopentanecarbohydrazide;
N′-(2-methylphenyl)-1-phenylcyclopentanecarbohydrazide;
1-methyl-N′-(2-methylphenyl)cyclohexanecarbohydrazide;
4-methoxy-N′-(2-methylphenyl)cyclohexanecarbohydrazide;
N′-(2-methylphenyl)-1-phenylcyclopropanecarbohydrazide;
N′-(2-methylphenyl)tetrahydrofuran-2-carbohydrazide;
N′-(2-methylphenyl)cyclobutanecarbohydrazide;
N′-(2-methylphenyl)cyclopentanecarbohydrazide
N′-(2-methylphenyl)cyclohexanecarbohydrazide;
2-methyl-N′-(2-methylphenyl)cyclohexanecarbohydrazide;
3-methyl-N′-(2-methylphenyl)cyclohexanecarbohydrazide;
4-methyl-N′-(2-methylphenyl)cyclohexanecarbohydrazide;
N′-(2-methylphenyl)cycloheptanecarbohydrazide;
1-methyl-N′-(2-methylphenyl)cyclopropanecarbohydrazide;
2-methyl-N′-(2-methylphenyl)cyclopropanecarbohydrazide;
1-acetyl-N′-(2-methylphenyl)piperidine-4-carbohydrazide;
N′-(2,3-dichlorophenyl)adamantane-1-carbohydrazide;
N′-(2-chlorophenyl)adamantane-1-carbohydrazide;
N′-phenyladamantane-1-carbohydrazide;
N′-(pentafluorophenyl)adamantane-1-carbohydrazide;
N′-(2,5-dichlorophenyl)adamantane-1-carbohydrazide;
N′-(2,4-dichlorophenyl)adamantane-1-carbohydrazide;
N′-(3,4-dichlorophenyl)adamantane-1-carbohydrazide;
N′-(4-fluorophenyl)adamantane-1-carbohydrazide;
N′-(4-methoxyphenyl)adamantane-1-carbohydrazide;
N′-(2,5-dimethylphenyl)adamantane-1-carbohydrazide;
N′-(4-cyanophenyl)adamantane-1-carbohydrazide;
N′-(2-fluorophenyl)adamantane-1-carbohydrazide;
N′-(3-fluorophenyl)adamantane-1-carbohydrazide;
N′-(4-methylphenyl)adamantane-1-carbohydrazide; and
N′-[3-(trifluoromethyl)phenyl]adamantane-1-carbohydrazide.
